(19) United States
(12) Patent Application Publication
(10) Pub. No.: US 2022/0395602 A1
(43) Pub. Date: Dec. 15, 2022

(54) MODULATED SCENT RELEASE DEVICE

(71) Applicant: Enviroscent, Inc., Atlanta, GA (US)

(72) Inventors: Christopher L. MCCLINTOCK, Bogart, GA (US); Eric MEHNERT, Lawrenceville, GA (US); Tamara KULLBACK, Atlanta, GA (US); Carolina Gomez MAINOR, Alpharetta, GA (US); Brady SCHROEDER, Milton, GA (US)

(73) Assignee: Enviroscent, Inc., Atlanta, GA (US)

(21) Appl. No.: 17/776,913

(22) PCT Filed: Nov. 14, 2019
(86) PCT No.: PCT/US2019/061576

§ 371 Date: May 13, 2022

(51) Int. Cl. A61L 9/12

(52) U.S. Cl. CPC A61L 9/127; A61L 2209/15; A61L 2209/133

(57) ABSTRACT

Described is a scent producing assembly having a self-contained module comprising an absorptive matrix infused with a volatile composition and a housing. The housing may include a receptacle shaped to receive the module, wherein the housing has a stowed position and a deployed position. In the stowed position, the receptacle may be contained within an interior of the housing. In the deployed position, the receptacle may be displaced out of the housing. The receptacle may move along an arcuate path when the receptacle is moved between the stowed and deployed positions.

### FIELD OF THE INVENTION

The field of the invention relates to systems and devices for producing modulated release of volatile olfactory or fragrance compounds that include articles formed of pulp base materials.

### BACKGROUND

Fragrance-releasing devices are well known and commonly used in household and commercial establishments to provide a pleasant environment for people in the immediate space. Further, aroma-driven experiences are well recognized to improve or enhance the general mood of individuals. In some instances, fragrances may trigger memories of experiences associated with the specific scent. Whether it is providing a pleasant environment, affecting a general demeanor, or triggering a nostalgic memory, a steady, long-lasting release of fragrance will ensure consumer and customer satisfaction.

Fragrance-release devices based on active diffusion are often based on liquid material being drawn up a wick to the heated tip of the wick and then being expelled from a chamber in the device that holds a finite supply of the liquid fragrance.

The evaporation rate of fragrance from the fragrance-release device is determined, at least in part, by the composition of the fragrance, where compositions containing more volatile compounds (e.g. “top” notes) will evaporate faster than those with less volatile compounds (e.g. “base” notes). A fragrance composition determines its character. As a result, changing the composition of the fragrance will affect the character. These typical devices require a high amount of very volatile solvent/diluent to help draw the fragrance up the wick. This results in a fragrance composition that is mostly solvent diluent, with the minor part of the composition actually being fragrance.

For these fragrances, there is a need to use the energy source to facilitate the release of fragrance from the fragrance-release device and provide a steady and long-lasting fragrance release. This energy source can be moved (in some models) up and down the top part of the wick to supposedly modulate the amount of fragrance coming off. However, in practice, many consumers notice no actual difference.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim.

According to certain embodiments of the present invention, a scent producing assembly comprises a self-contained module comprising an absorptive matrix infused with a volatile composition, and a housing comprising a receptacle shaped to receive the module. The receptacle may have a stowed position, wherein the receptacle is contained within an interior of the housing, and a deployed position, wherein the receptacle is displaced outside of the housing. The receptacle may be moved between the stowed and deployed positions, such that the receptacle moves along an arcuate path.

In some embodiments, the housing further comprises an opening along an edge and movement of the receptacle between the stowed and deployed positions moves through the opening. The receptacle may be attached to the housing via a single point and during movement between the stowed and deployed positions, the receptacle rotates about the pivot point. In the stowed position, an exterior surface of the receptacle may form an exterior edge of the housing. The receptacle may further comprise a locking mechanism such that the receptacle is lockable in the stowed position.

According to certain embodiments of the present invention, a scent producing assembly comprises a self-contained module comprising an absorptive matrix infused with a volatile composition, and a housing comprising a housing exterior, a housing interior, an opening formed along a side of the housing exterior, and a receptacle shaped to receive the module. The receptacle may have a stowed position and a deployed position. In the stowed position, the receptacle may be disposed within the housing interior. In the deployed position, the receptacle may be disposed outside of the housing interior. When the receptacle is moved between the stowed position and the deployed position, the receptacle may move through the opening along an arcuate path.

In some embodiments, the receptacle comprises a first end and a second end, wherein the first end is attached to the housing at a pivot point and the second end comprises an extension for locking the receptacle in the stowed position. When the receptacle is in the stowed position, an exterior surface of the receptacle may form an edge of the housing exterior. During movement between the stowed and deployed positions, the receptacle may pivot about the pivot point and the second end may travel along the arcuate path. Moreover, during movement between the stowed and deployed positions, the self-contained module may be contained within the receptacle.

In some embodiments, the self-contained module is formed of at least one of recyclable materials or biodegradable materials.

According to certain embodiments of the present invention, a scent producing assembly comprises a self-contained module comprising an absorptive matrix infused with a volatile composition, a housing comprising an interior, wherein the interior is hollow, and a receptacle shaped to receive the self-contained module. The receptacle may be connected to an edge of the housing at a pivot point. The self-contained module may be moved between the interior of the housing and an exterior of the housing via the receptacle. During movement of the self-contained module between the interior of the housing and the exterior of the housing, the receptacle may pivot about the pivot point and move along an arcuate path.

In some embodiments, the housing exterior comprises a first side and a second side, wherein each of the first side and the second side comprises openings on a first side and a second side such that air can freely pass through the housing.

In some embodiments, the housing may further comprise an opening along an edge of between the first side and the second side. The opening may provide access to the interior of the housing. When the receptacle is disposed within the interior of the housing, an outer edge of the receptacle may form an exterior edge of the housing. During movement of the self-contained module between the interior and the exterior of the housing, the receptacle may move through the opening.

In some embodiments, the receptacle further comprises a locking mechanism at one end opposite the pivot point such that the receptacle is lockable when the self-contained module is disposed within the interior of the housing.

In some embodiments, the scent producing further comprises an attachment mechanism disposed on the housing exterior.

### BRIEF DESCRIPTION OF THE DRAWINGS

In the following detailed description, embodiments of the invention are described referring to the following figures:

FIG. 1 is an image of a mold used to form a absorptive matrix, according to certain embodiments of the present invention.

FIG. 2 is a top view of an article formed from the absorptive matrix formed with the mold of FIG. 1.

FIG. 3 is a side view of the article of FIG. 2.

FIG. 4 is a top view of an article formed from a absorptive matrix with a divider, according to certain embodiments of the present invention.

FIG. 5 is a side view of an article formed from a absorptive matrix with a divider in which the top and bottom surfaces of the divider are covered by pulp material, according to certain embodiments of the present invention.

FIG. 6 is a side view of an article formed from a absorptive matrix with a divider in which the top surface of the divider are covered by pulp material, according to certain embodiments of the present invention.

FIG. 7 is a side view of an article formed from a absorptive matrix with a divider in which the top and bottom surfaces of the divider are not covered by pulp material, according to certain embodiments of the present invention.

FIG. 8 is a side view of an article formed from a absorptive matrix with a divider comprising a backing layer, according to certain embodiments of the present invention.

FIG. 9 is a top view of an article formed from a absorptive matrix with a divider comprising multiple zones, according to certain embodiments of the present invention.

FIG. 10 is a flow diagram of a multi-step molding process, according to certain embodiments of the present invention.

FIG. 11 is a side view of an article formed from a absorptive matrix with complex surface geometry, according to certain embodiments of the present invention.

FIG. 12 is a side view of an article formed from a absorptive matrix with complex surface geometry, according to certain embodiments of the present invention.

FIG. 13 is a top view of an article formed from a absorptive matrix with an attachment element, according to certain embodiments of the present invention.

FIG. 14 is a top view of an article formed from a absorptive matrix with an opening, according to certain embodiments of the present invention.

FIG. 15 is a top view of an article formed from a absorptive matrix with a plurality of openings for addition of other materials, according to certain embodiments of the present invention.

FIG. 16 is a top view of the article of FIG. 15 with the other materials incorporated into the plurality of openings.

FIG. 17 is a side view of two articles each formed from absorptive matrices being joined, according to certain embodiments of the present invention.

FIG. 18 is a side view of the articles of FIG. 17 joined.

FIG. 19 is a side view of an article formed from a absorptive matrix with a capillary system for introduction of volatile compositions into the absorptive matrix.

FIG. 20 is a front perspective view of a scent producing assembly according to certain embodiments of the present invention.

FIG. 21 is a top perspective view of the scent producing assembly of FIG. 20.

FIG. 22 is another perspective view of the scent producing assembly of FIG. 20.

FIG. 23 is front elevation view of the scent producing assembly of FIG. 20.

FIG. 24 is a side elevation view of the scent producing assembly of FIG. 20.

FIG. 25 is a side perspective view of the scent producing assembly of FIG. 20.

FIG. 26 is a rear elevation view of the scent producing assembly of FIG. 20.

FIG. 27 is a perspective view of the scent producing assembly of FIG. 20 with the receptacle transitioning to the deployed position.

FIG. 28 is a front view of the scent producing assembly of FIG. 20 with the receptacle in the deployed position.

FIG. 29 is a rear perspective view of scent producing assembly of FIG. 20 with the receptacle in the deployed position.

FIG. 30 is a front perspective view of the scent producing assembly of FIG. 20 with the receptacle in the deployed position.

FIG. 31 is a side perspective view of the scent producing assembly of FIG. 20 with the receptacle in the deployed position and the attachment device removed.

FIG. 32 is a side perspective view of the scent producing assembly of FIG. 20 with the receptacle in the deployed position and the attachment device removed.

FIG. 33 is a schematic illustrating the movement of a volatile composition across an internal structure of a base material and a modulating coating over time, according to certain embodiments of the present invention.

FIG. 34 is a microphotograph image of a cross-section of a sample of a three-dimensional pulp object comprising a low density absorptive matrix, according to certain embodiments of the present invention.

FIG. 35 is a microphotograph image of a cross-section of a sample of a three-dimensional pulp object comprising a high density absorptive matrix, according to certain embodiments of the present invention.

FIG. 36 is a microphotograph image of a cross-section of a sample of a three-dimensional pulp object with both high density pulp material and low density pulp material, according to certain embodiments of the present invention.

FIG. 37 is a microphotograph low-angle reflected light image of a cross-section of a sample of a three-dimensional pulp object comprising a low density absorptive matrix after iodine staining, according to certain embodiments of the present invention.

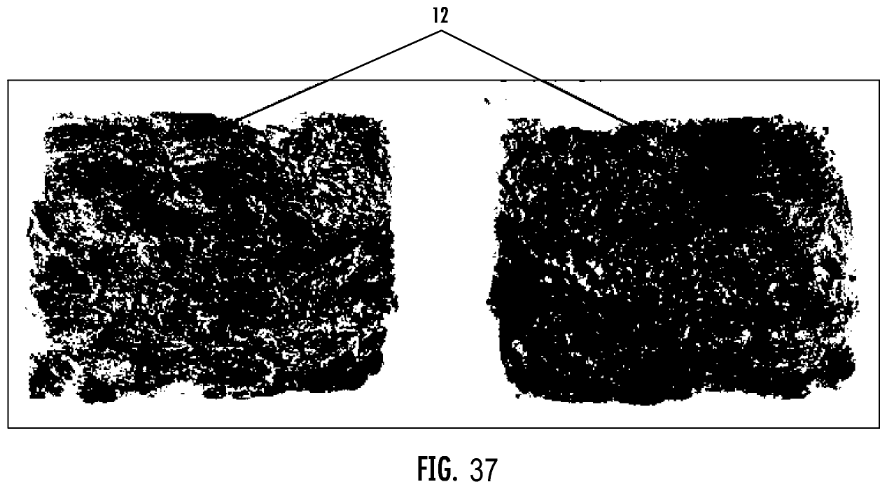

FIG. 38 is a microphotograph low-angle reflected light image of a cross-section of a sample of a three-dimensional pulp object comprising a high density absorptive matrix after iodine staining, according to certain embodiments of the present invention.

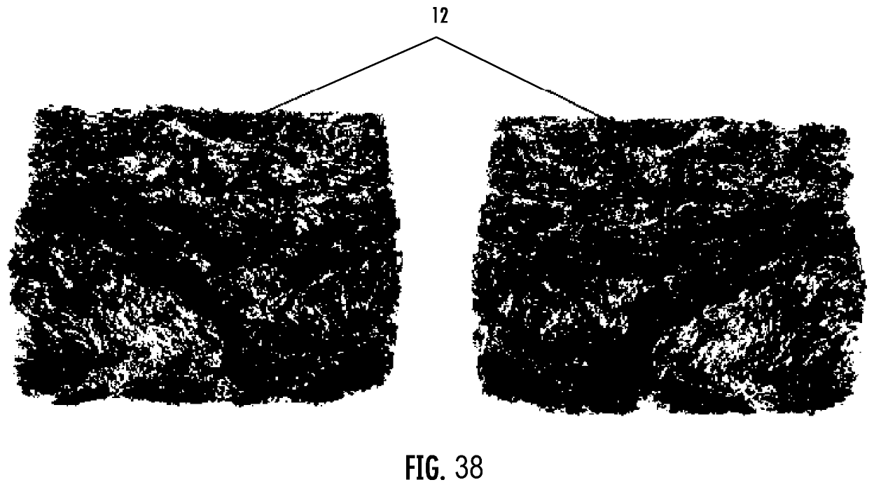

FIG. 39 is a high resolution image of the cross-section of the low density sample of FIG. 37.

FIG. 40 is a high resolution image of the cross-section of the high density sample of FIG. 38.

FIG. 41 is a graph showing weight loss data for two density zones according to certain embodiments of the present invention.

FIG. 42 is a graph showing weight loss data for an article according to certain embodiments of the present invention.

FIG. 43 is a graph showing hedonic data for a scent producing assembly according to certain embodiments of the present invention.

FIG. 44 is a graph showing weight loss data for a scent producing assembly according to certain embodiments of the present invention.

### DETAILED DESCRIPTION

The subject matter of embodiments of the present invention is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

According to certain embodiments of the present invention, an article 10 comprises a absorptive matrix 12. The article 10 may be a three-dimensional object including for example, a box, a cube, a sphere, a cylinder, or any other appropriate shape. In some embodiments, the article 10 is formed into a sheet (i.e., where the thickness is relatively small compared to the surface area) such that portions can be cut into smaller individual parts. The article 10 may be formed so as to be biodegradable and/or recyclable. Similarly, the article 10 may be encased in a scent producing assembly 2000 that is recyclable and/or biodegradable.

A. Absorptive Matrix

The absorptive matrix 12 may comprise an internal structure 20 comprising a plurality of pores 22 that are configured to provide locations for the volatile composition 24 to be stored therein and released therefrom, which is described in detail below.

The absorptive matrix 12 may comprise natural and/or synthetic pulp compositions; pulp compositions combined with other products, including but not limited to paper, cellulose, cellulose acetate, pulp lap, cotton linters, biological plant-derived materials (from living plants), synthesized pulp compositions, and mixed pulps; polymer material; porous material; and/or extrudate.

As known in the art, pulp is primarily a collection of fibers with other components of the source material, wherein the fibers are derived from a natural or synthetic source material, for example, biological plants (natural) or petroleum-based synthesis products (synthetic). Pulp may be produced from various types of woods using any one of several known pulping techniques. The pulp may be from hardwoods, softwoods, or mixtures thereof. The pulp may also be produced from bamboo, sugarcane, and other pulp sources. The pulp may also be made from recycled materials, and comprises recovering waste paper and remaking it into new products.

In certain embodiments, the number and/or size of the plurality of pores 22 (i.e., porosity) within the absorptive matrix 12 may be controlled by the compactness and/or size of the fibers and/or particles that form the internal structure 20. For example, in certain embodiments of the absorptive matrix 12 that comprise fibers, voids between the fibers form tiny air passages throughout the internal structure 20. The compactness of the fibers affects the degree in which the absorptive matrix 12 allows gas or liquid to pass through it. For example, porosity may affect absorbency, uptake, and/or load amount of volatile compositions, or may affect the rate of release of such substances. Porosity and/or absorbency of the absorptive matrix 12 may be affected by adding other materials, such as additives to the matrix material 12 as it is being formed from a composition, such as pulp or any other composition described above, so that the additives are located within the internal structure 20 of the absorptive matrix 12 after formation.

The porosity of an absorptive matrix 12 that comprises pulp may be affected at any stage of the pulp production process. An increased level of fiber refining causes the fibers to bond together more strongly and tightly, making the pulp material denser, thereby reducing the network of air passages and the porosity. The porosity of the absorptive matrix 12 may also be controlled using other compression methods, which are described in detail below.

The porosity of the absorptive matrix 12 is measured quantitatively as either the length of time it takes for a quantity of air to pass through a sample, or the rate of the passage of air through a sample, using either a Gurley densometer (in the first case) or a Sheffield porosimeter (in the second case). With the Gurley densometer, the porosity is measured as the number of seconds required for 100 cubic centimeters of air to pass through 1.0 square inch of a given material at a pressure differential of 4.88 inches of water, as described in ISO 5646-5, TAPPI T-460, or TAPPI T-536.

The porosity may affect how completely and how quickly the volatile composition 24 is absorbed into the absorptive matrix 12, as such absorption may occur primarily by capillary action. For example, an absorptive matrix 12 with high porosity may have increased absorbency of the volatile composition 24. As an example relating porosity to standard test methods for sheets of paper, the porosity of the absorptive matrix 12 may range from 0.01 Gurley second-100 Gurley seconds, and all ranges therein. In certain embodiments where there are multiple layers of absorptive matrix 12, the porosity may range from 0.01 Gurley second-20 Gurley seconds. The volatile composition 24 may be applied to the absorptive matrix 12 in the form of a film or a coating, or as a treatment integrated into the internal structure 20 of the absorptive matrix 12. The difference in porosities affects the release rate of the volatile composition 24, as the lower porosity has a lower release rate, whereas the higher porosity has a higher release rate. Having a higher porosity in one portion of the absorptive matrix 12 (such as inner layer or inner ply) compensates for the fact that the volatile composition 24 has to travel through more layers/plies to reach the outside of the absorptive matrix 12. It is also noted that the density of the absorptive matrix 12 affects the internal reservoir of the absorptive matrix 12 (i.e., the capacity to absorb the volatile composition 24).

In some embodiments, different thicknesses of the absorptive matrix 12 may have different amounts of compression applied during the manufacturing process such that the resultant absorptive matrices 12 may have varying densities, porosities, and absorbencies.

Additional description of absorptive matrices, porosity, pulp concentrations, related embodiments, etc. may be found in U.S. Publication No. 2011/0262377 and U.S. Publication No. 2019/0231919, the entire contents of each of which are incorporated herein by reference.

In certain embodiments, the porosity of the absorptive matrix 12 may be controlled such that the absorptive matrix 12 is configured with varying porosity zones 1202. In some embodiments, the porosity zones 1202 may be formed by changing the compactness of the fibers within the absorptive matrix 12.

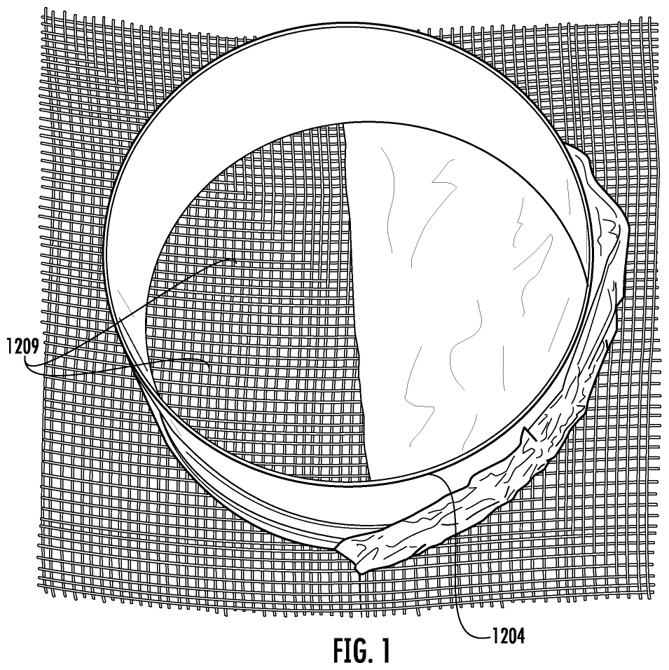

For example, the absorptive matrix 12 may be formed within a mold 1204, as shown in FIG. 1. The mold 1204 is configured to form an absorptive matrix 12 having at least one high porosity zone 1206 and at least one low porosity zone 1208.

The absorptive matrix 12 positioned over the portion of the mold 1204 having a plurality of apertures 1209 in the base surface comprises the low porosity zone 1208. When pressure is uniformly applied to the absorptive matrix 12, more water is removed from that zone of the absorptive matrix 12 via the drainage apertures 1209. As a result, the low porosity zone 1208 will have greater fiber compactness (and thus a greater density).

In contrast, the absorptive matrix 12 positioned over the portion of the mold 1204 with the solid base surface comprises the high porosity zone 1206. When pressure is uniformly applied to the absorptive matrix 12, less water is removed from that zone of the absorptive matrix 12 because there is no additional drainage mechanism to assist with water removal. As a result, the high porosity zone 1206 will have less fiber compactness (and thus a lower density).

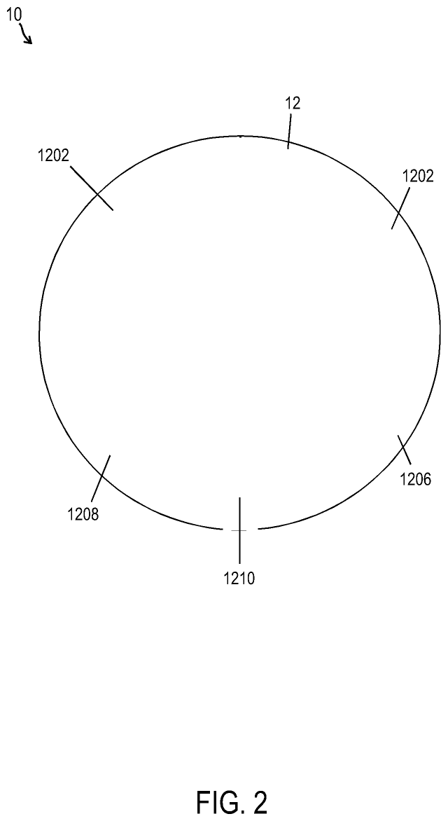

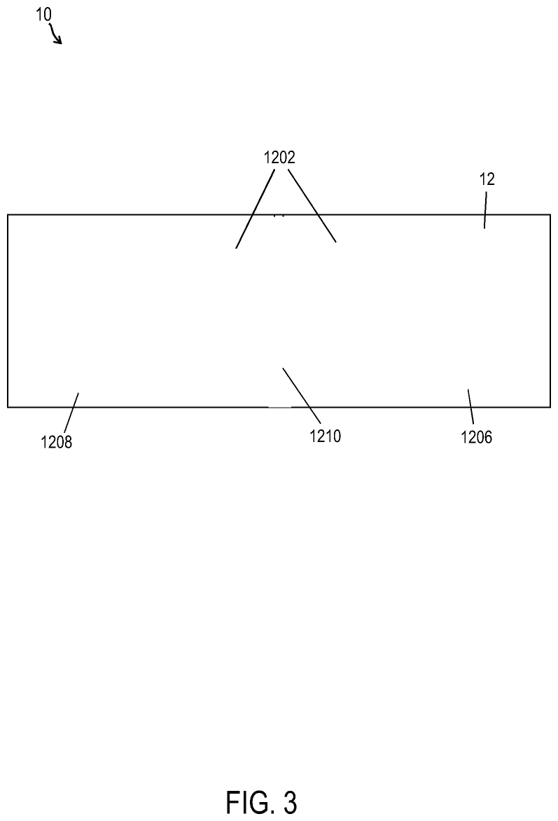

As best illustrated in FIGS. 2-3, there may be transitional porosity zones 1210 between the high porosity zone 1206 and the low porosity zone 1208, in which the fiber compactness gradually changes. When the volatile composition(s) 24 are infused into zones 1206, 1208 of the absorptive matrix 12, a certain amount of wicking of the volatile composition(s) 24 may occur through the transitional porosity zones 1210. Although some drawings show the absorptive matrix 12 with two porosity zones, the absorptive matrix 12 may include a plurality of zones with different properties (including porosity, density, or other properties). The absorptive matrix 12 may have any number of zones that each include different properties.

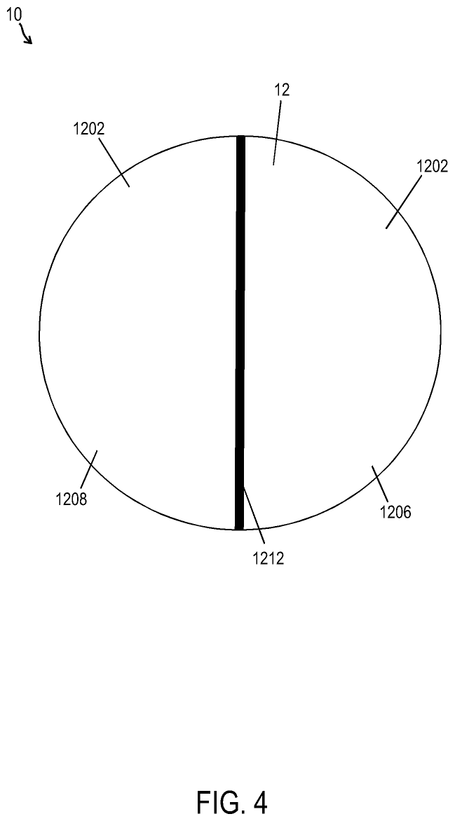

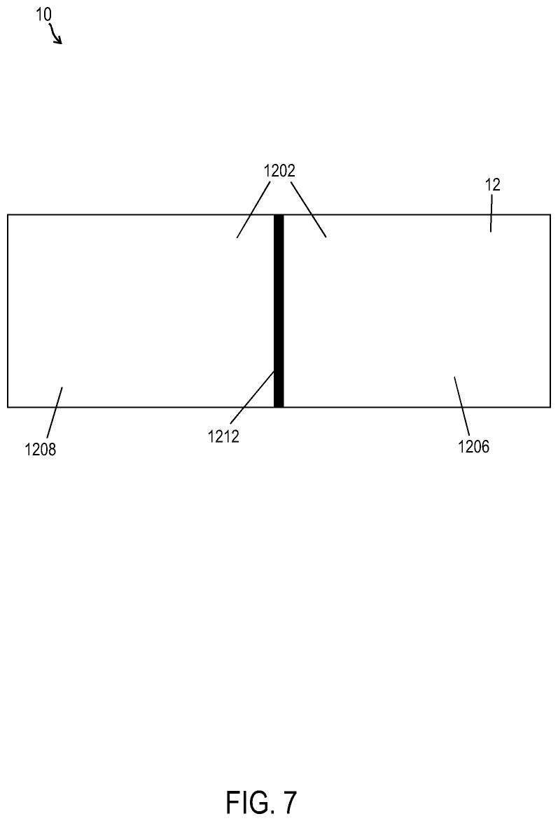

In further embodiments, as best illustrated in FIGS. 4-9, a divider 1212 may be positioned, or at least partially embedded within the absorptive matrix 12. To position the divider 1212 within the absorptive matrix 12, the divider 1212 may be positioned within the mold 1204 when the absorptive matrix material is introduced into the mold 1204. The divider 1212 may be shaped to separate the zones 1206 and 1208 so as to eliminate some or substantially all of the transitional porosity zones 1210, as well as some or substantially all of the wicking of the volatile composition 24 between the various porosity zones 1202.

In some embodiments, the absorptive matrix material with a lower concentration of fibers may be added to the high porosity zone 1206, and the absorptive matrix material with a higher concentration of fibers may be added to the low porosity zone 1208. When pressure is uniformly applied to the mold 1204, the high porosity zone 1206 will have less fiber compactness (and thus a lower density) than the low porosity zone 1208. When pressure is applied to compact the mold 1204 to a uniform distance, the low porosity zone 1208 will have greater fiber compactness (and thus a higher density) due to a greater number of fibers per volume, than the high porosity zone 1206.

Alternatively, an absorptive matrix 12 having a uniform concentration of fibers may be added to both zones 1206, 1208. More pressure may be applied to the low porosity zone 1208, thereby compressing it more to reduce the porosity (i.e., by compacting the fibers more and increasing the density). In contrast, less pressure may be applied to the high porosity zone 1206, thereby compressing it less than the low porosity zone 1208.

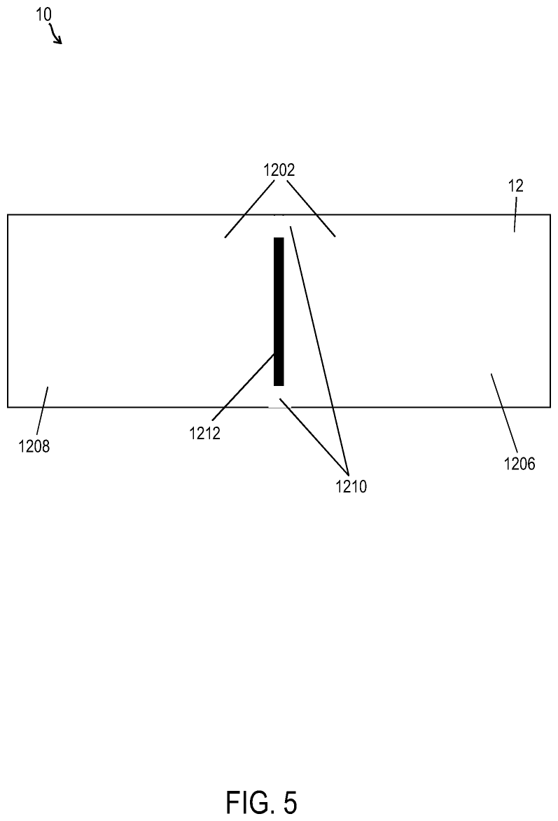

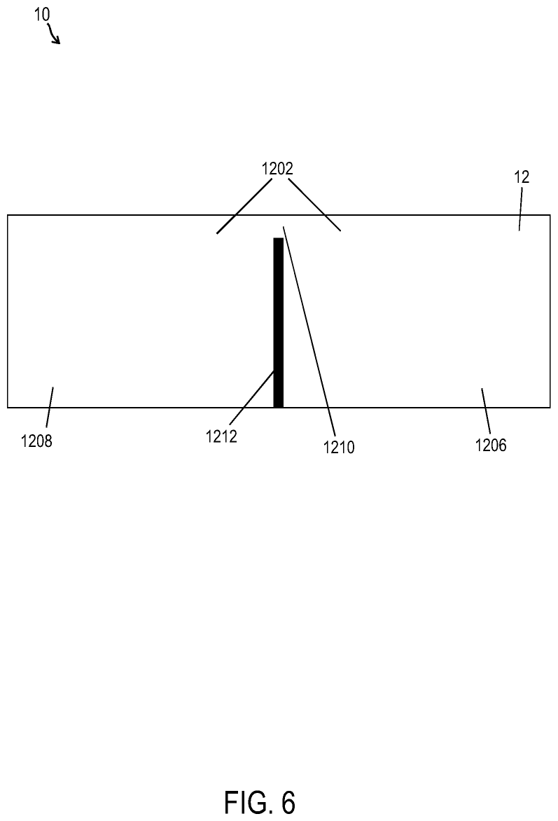

As best illustrated in FIGS. 5-6, the divider 1212 may be shaped so as to be at least partially embedded within the absorptive matrix 12. In these embodiments, a portion of the absorptive matrix material may extend over an upper (FIGS. 5-6) and/or lower (FIG. 6) surface of the divider 1212 so that the divider 1212 is not visible through the overlapping absorptive matrix material. When the volatile composition(s) 24 are infused into zones 1206, 1208 of the absorptive matrix material, a certain amount of wicking of the volatile composition(s) 24 may occur through the overlapping absorptive matrix material.

In other embodiments, as best illustrated in FIGS. 4 and 6-9, the divider 1212 may be shaped so as to form at least a portion of a visible surface of the article 10. In these embodiments, the divider 1212 may be shaped so as to form a portion of a decorative design or other aesthetically appealing surface treatment of the article 10.

In further embodiments, the porosity zones 1202 may be formed by introducing varying amounts of a pore-forming agent such as a gas or gas-forming material. The gas or gas-forming material may be introduced into the absorptive matrix 12 prior to or after introduction into the mold 1204. Examples of gas-forming materials include solids, volatile liquids, chemical reagents, such as calcium carbonate and acid, thermally decomposable materials which will cause evolution of a gas by, for example, decomposition of bicarbonate, or biological agents, such as dextrose and yeast. Different amounts of gas or gas-forming materials may be introduced into each zone 1206, 1208, thereby producing zones with differing porosities, even if the fiber content of each zone is approximately the same. For example, the high porosity zone 1206 may be infused with a larger amount of a gas or gas-forming material, thereby having a greater porosity, while the low porosity zone 1208 may be infused with a lesser amount of a gas or gas-forming material, thereby having a lower porosity.

In further embodiments, zones 1206 and 1208 may be formed in completely separate molds 1204 using any of the above techniques (i.e., fiber compactness, infusion of gas or gas-forming materials, refining, additives, or any other porosity-controlling method described above) to adjust the porosity of zone 1206 relative to the porosity of zone 1208.

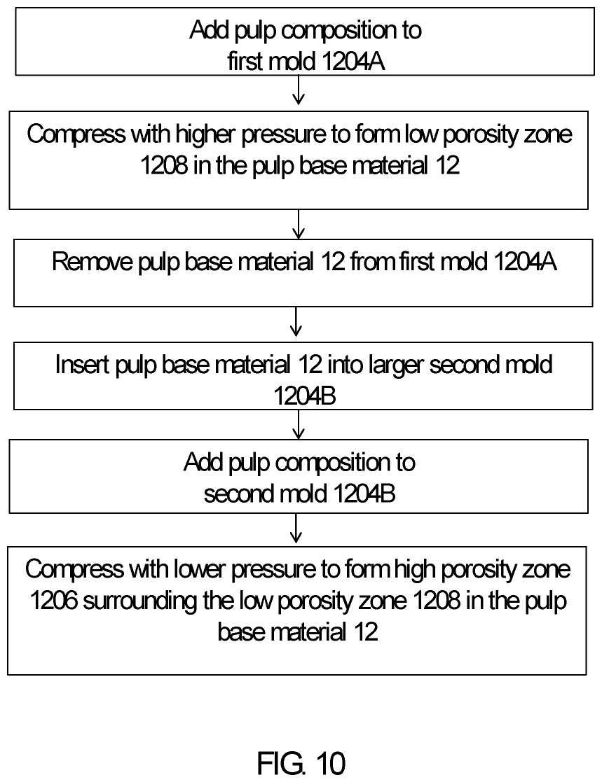

Furthermore, as described in FIG. 10, the absorptive matrix 12 may be formed using at least two molding steps. In the first step, the absorptive matrix material is added to a first mold 1204A, which is then compressed using a higher pressure (in the range of 0.1 lb/in² to 100 lb/in²) to form the low porosity zone 1208. The absorptive matrix 12 is removed from the first mold 1204A, and then inserted into a second mold 1204B having a larger volume than the first mold 1204A. Additional absorptive matrix material is then added to the second mold 1204B to surround the absorptive matrix 12 from the first mold 1204A. The material inside mold 1204B is then compressed using a lower pressure (in the range of 0.1 lb/in² to 100 lb/in²) to form the high porosity zone 1206. This technique forms a absorptive matrix 12 having discrete porosity zones 1202 without the transitional porosity zones 1210 forming between the porosity zones 1202 and also without the need for a divider 1212 to separate the zones. Additionally, a treatment may be applied to the low porosity zone 1208 before additional absorptive matrix material is added to the second mold 1204B to maintain the shape and/or density of the low porosity zone 1208 after addition of the additional absorptive matrix material. Examples of the treatment include, but are not limited to wet strength agents, binders, wax, starch, sizing, cross-linking reagents, and/or any other suitable agent.

In some embodiments, the high porosity zone 1206 has a density of approximately 0.6 g/cm³ to 0.9 g/cm³ and the low porosity zone 1208 has a density of approximately 1.0 g/cm³ to 1.2 g/cm³. In certain embodiments, the high porosity zone 1206 has a density of approximately 0.7 g/cm³ to 0.75 g/cm³ and the low porosity zone 1208 has a density of approximately 1.05 g/cm³ to 1.1 g/cm³. The entire absorptive matrix 12 may have a density ranging between 0.30 g/cm³ to 0.75 g/cm³ and, more specifically, between 0.40 g/cm³ to 0.65 g/cm³. As the density of the absorptive matrix 12 increases, the maximum amount of fragrance (liquid, such as volatile composition 24) that can be absorbed into the absorptive matrix 12 decreases. In some embodiments, after liquid has been absorbed, the high porosity zone 1206 has a percent fragrance load of approximately 50%-54% and the low porosity zone 1208 has a percent fragrance load of approximately 42%-46%. In some embodiments, after liquid has been absorbed, the high porosity zone 1206 and the low porosity zone 1208 have a percent fragrance load of approximately 30%-70%. In certain embodiments, after liquid has been absorbed, the high porosity zone 1206 has a percent fragrance load of approximately 51.5%-52.5% and the low porosity zone 1208 has a percent fragrance load of approximately 43.5%-44.5%.

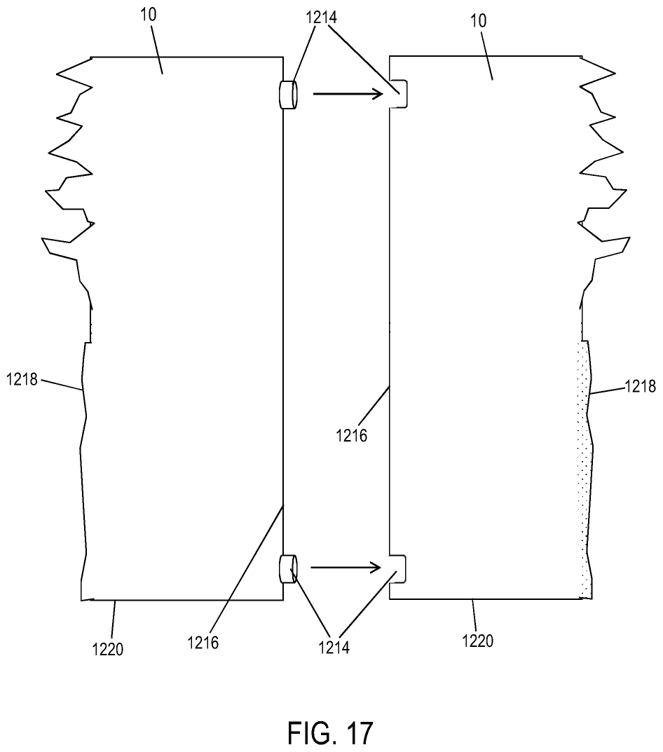

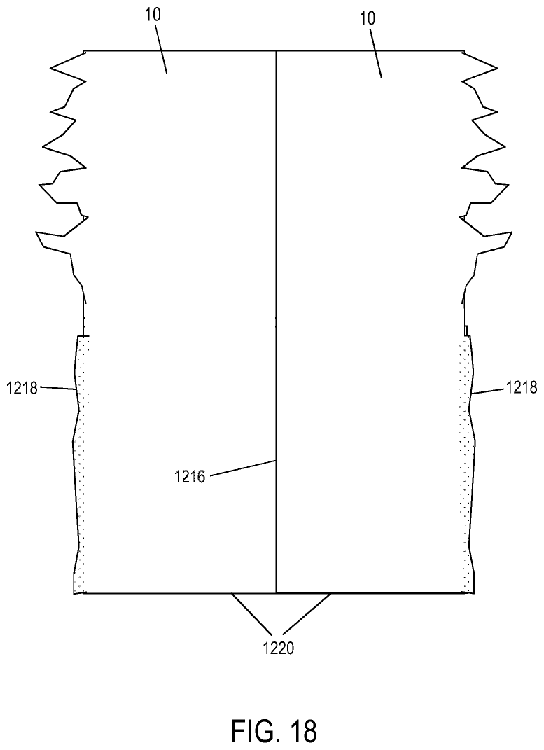

In the embodiments where the divider 1212 is shaped so as to completely eliminate any overlapping absorptive matrix 12 between the zones 1206, 1208 and/or where the zones 1206,1208 are formed in different molds, joining mechanisms 1214 between the zones 1206, 1208 may be used to discrete units of the absorptive matrix 12 into the article 10, as illustrated in FIGS. 17-18.

Examples of suitable joining mechanisms 1214 may include, but are not limited to, any suitable chemical fasteners such as adhesives, coatings, wax, starch, and gums, and/or any suitable mechanical fasteners such as male/female clips, anchors, hook and loop fasteners, pins, screw-type fasteners, impregnation-type fasteners, and magnets. These mechanical fasteners may, in certain embodiments, be part of the molding process itself and may be made out of pulp.

FIG. 17 illustrates an example of joining mechanisms 1214 that may be used. In certain embodiments, the joining mechanisms 1214 may be included in the mold when the absorptive matrix 12 is formed. In other embodiments, the joining mechanisms 1214 may be added to the zones 1206, 1208 after the molding process is completed.

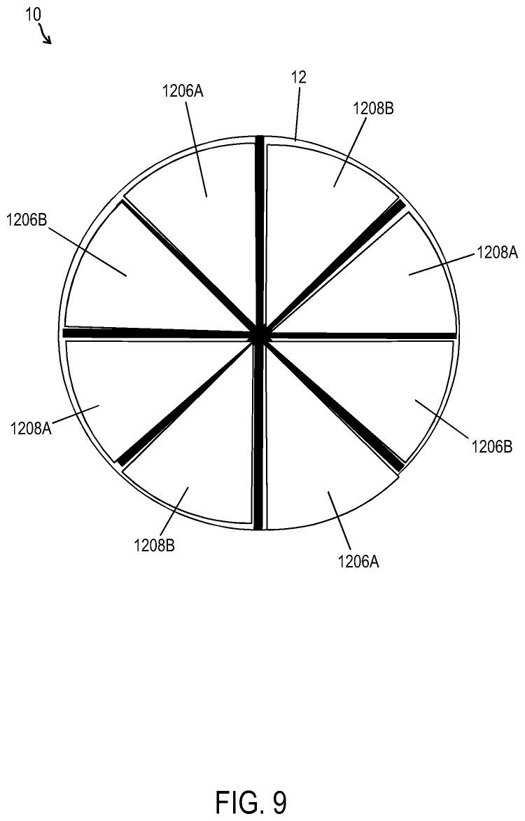

While the above description of the absorptive matrix 12 focused on two porosity zones 1206, 1208, the embodiments are by no means so limited. For example, the above techniques and mechanisms may be used to form a absorptive matrix 12 having any suitable number of zones, including but not limited to three, four, five, six, or more zones. As illustrated in FIG. 9, the absorptive matrix 12 may include eight zones: zones 1206A having the highest porosity, 1206B having high porosity, 1208A having low porosity, and 1208B having the lowest porosity.

In some embodiments, the article 10 is a uniform sheet which is then pressed into a slimmer sheet. The slimmer sheet would have higher density towards the edges. Also, other materials, such as starch and/or silica, can be added to the pulp directly or as a coating. The sheet may then be cut (e.g., using a die) into smaller pieces having particular shapes. In some cases, the sheet or the smaller pieces can be molded into particular shapes.

Furthermore, the zones may have any suitable shape, which includes but is not limited to wedge or pie shapes, rectilinear, elliptical, circular, or any suitable type of simple or complex geometry. Furthermore, while the zones 1206, 1208 have been described as being formed with different porosities, the person of ordinary skill in the relevant art will understand that the zones 1206, 1208 may be formed of the same or similar porosities using any of the forming or joining techniques discussed above.

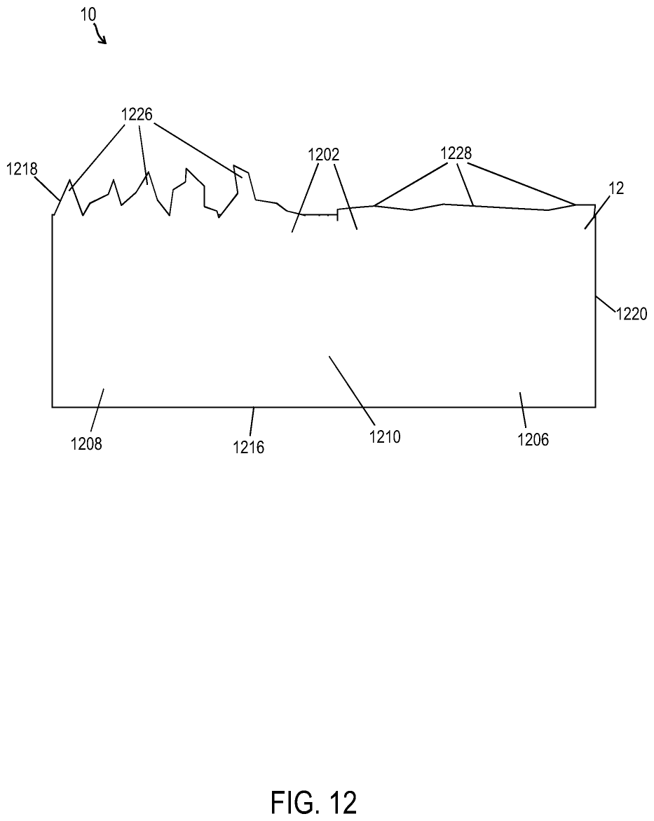

Furthermore, as best illustrated in FIGS. 2, 11-12, and 17-18, the zones may be formed with a relatively smooth interlocking surface 1216 for joining with other zones, while also having a very rough or complex exterior-facing surface 1218 that may include many peaks 1226 that form the outer surface of the absorptive matrix 12.

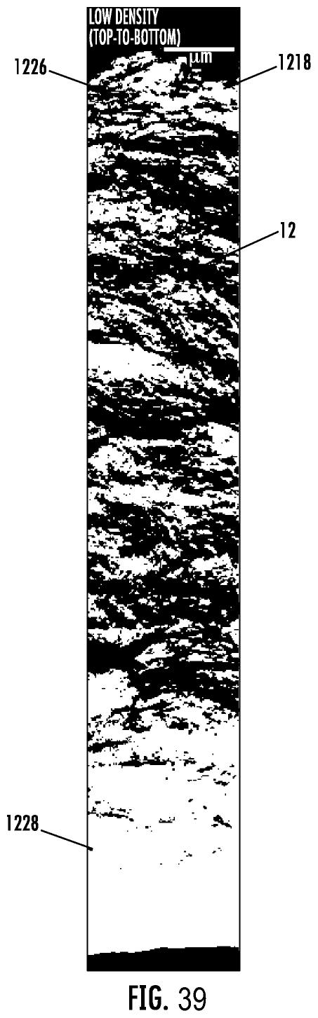

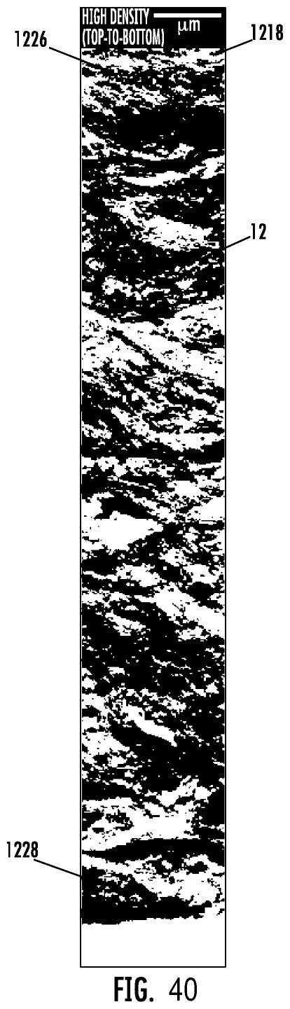

In some embodiments, the complex geometry of the exterior-facing surface 1218 may provide additional release rate control. For example, as shown in the attached microphotographs in FIGS. 39 and 40, the absorptive matrix 12 contains mini-variations in absorptive matrix materials that are located within peaks 1226 that are located on the surface 1218. The shape of the peaks 1226 causes the absorptive matrix material fibers to become more highly concentrated at a micro-scale in these areas, whereas the valleys or flatter regions 1228 are configured for better fiber dispersion at a micro-scale. As a result, there are variations in release rates from peak areas 1226 as compared to the flatter regions 1228. Additionally, as explained in more detail below, the different surface areas of the peaks 1226 and the valleys or flatter regions 1228 will also provide release rate control. Thus, the surface geometry may be configured to provide more peaks 1226 within the zone 1206 to further enhance the release rate of the “base notes,” while using a smoother surface texture within zone 1208 to further regulate the release rate of the “top notes.” Thus, the release rate can be tailored by density and/or surface area differences.

The location and concentration of the peaks 1226 also enhances the directionality of the release of the volatile composition 24. For example, the peaks 1226 act as small three-dimensional emitters, thus allowing the volatile composition 24 to emit from the raised surface of the peak 1226 in all directions. In contrast, the flatter regions 1228 tend to emit in more limited directionality because there is less surface area that faces in a range of directions. The range of emitting directionality provided by the peaks 1226 and flatter regions 1228 may be optimized and tied with locations of certain volatile compositions 24 within the absorptive matrix 12. The surface geometry may be designed to work in conjunction with porosity zones 1202 and/or with a absorptive matrix 12 having a relatively uniform porosity.

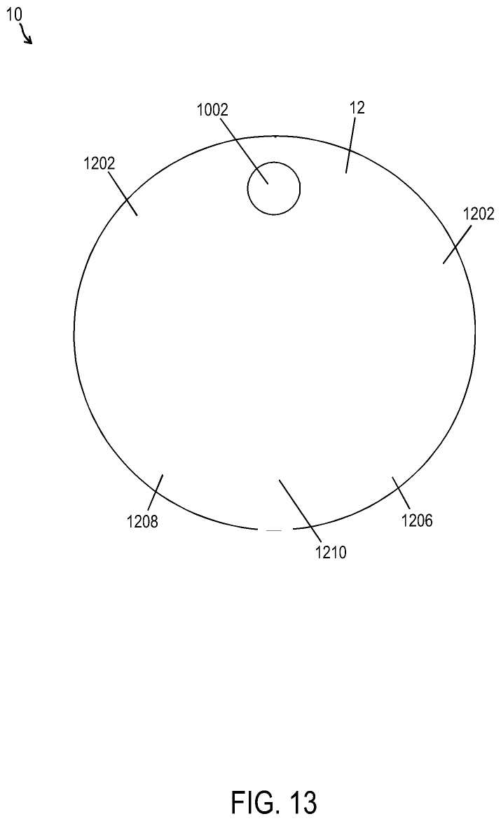

As shown in FIG. 13, the article 10 may include at least one attachment element 1002 for securing or holding the article 10. The attachment element 1002 may be a hole, a protrusion, a hook, a male/female clip, an anchor, a hook and loop fastener, a pin, a screw-type fastener, an electrical plug, a magnet, and/or any other appropriate object for securing the article 10. The attachment element 1002 may be formed in or attached to the article 10 after the absorptive matrix 12 has been molded. The attachment element 1002 may also be connected to or formed as part of the divider 1212 or other structure that is placed into the mold 1204 with the absorptive matrix material so that the attachment element 1002 is at least partially embedded within the absorptive matrix 12.

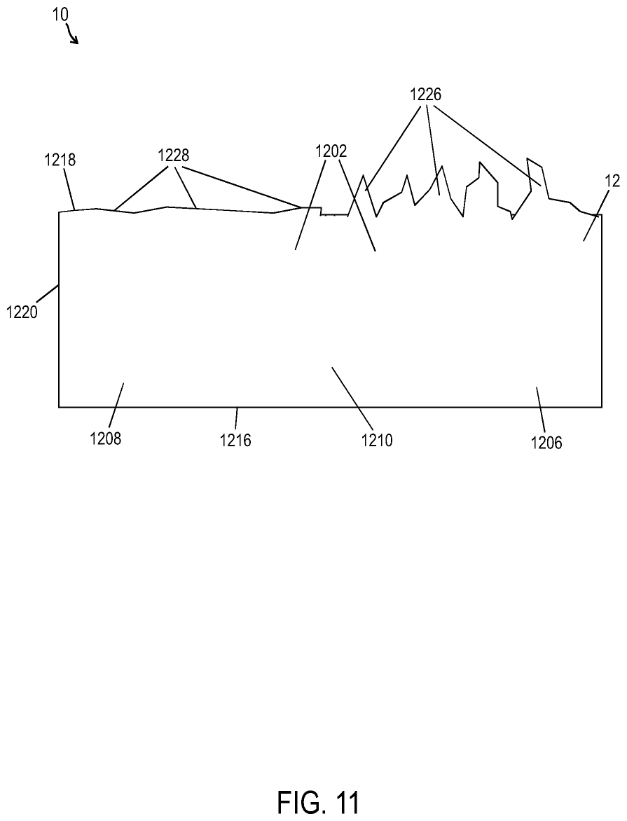

In some embodiments, as best illustrated in FIGS. 11-12 and 17-18, the article 10 may include an externally-facing smooth surface 1220 that forms a support surface to hold the article 10 in an upright position when positioned on another surface such as a table, desk, counter, window sill, etc.

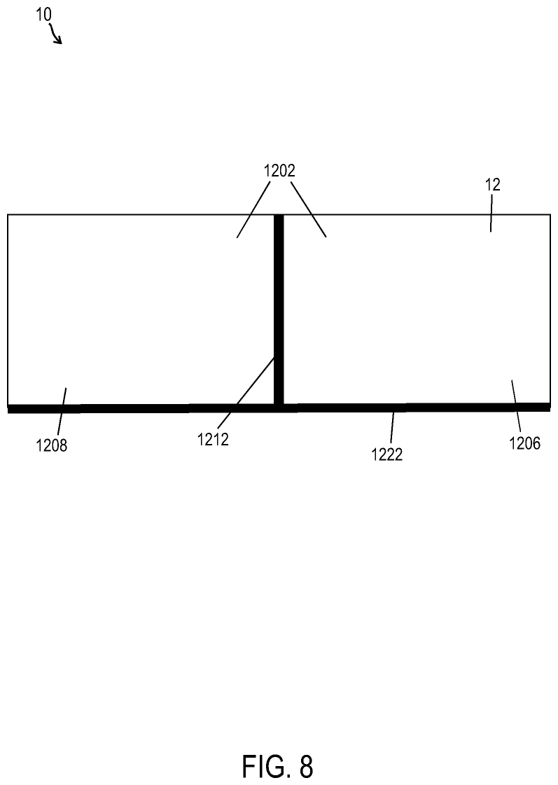

In other embodiments, as best illustrated in FIG. 8, the article 10 may include a backing layer 1222 that is applied to at least one surface of the article 10. The backing layer 1222 may be formed of any material that does not absorb or transmit the volatile composition 24 so as to prevent contact between the article 10 and other surfaces. Suitable materials include but are not limited to metal, metalized films, ceramic, glass, glazed ceramics, plastic, polymers, and any other impervious material.

The backing layer 1222 may be applied to the article 10 after the absorptive matrix 12 has been molded using any suitable chemical fasteners such as adhesives, coatings, wax, starch, gum and/or any suitable mechanical fasteners such as snap-fit design, male/female clips, anchors, hook and loop fasteners, pins, screw-type fasteners, impregnation-type fasteners, roughness or compatibility of the surface to bind fibers, and magnets.

In certain embodiments, as best illustrated in FIG. 8, the backing layer 1222 may also be connected to or formed as part of the divider 1212 or other structure that is placed into the mold 1204 with the absorptive matrix material so that the backing layer 1222 forms an exterior surface of the base pulp material 12. In some cases, the backing layer 1222 forms at least a portion of cover, a holder, or other appropriate component for the article 10. Such a configuration can help prevent direct contact between the material of the article 10 and the consumer's skin during handling of the article 10 and/or the replaceable module 2050 (described below). The backing layer 1222 may be a temporary or disposable component and/or may part of the packaging for the article 10 or the replaceable module 2050.

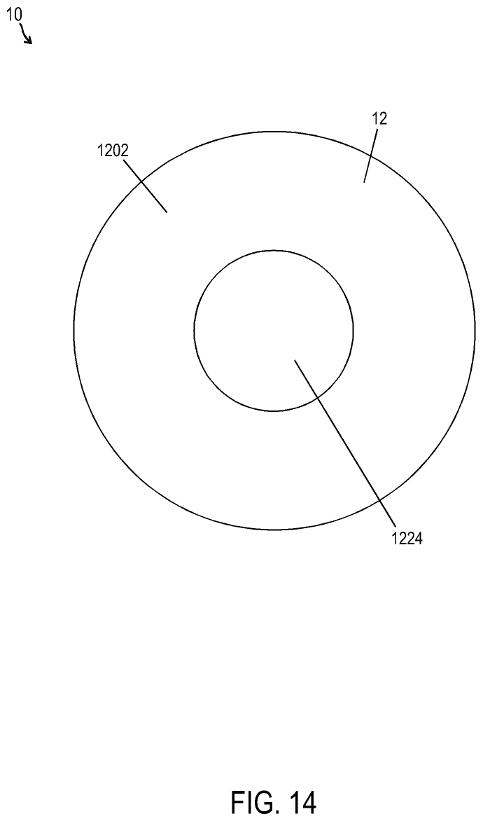

In further embodiments, as best illustrated in FIGS. 14-16, the article 10 may further include a dowel or other opening 1224 that extends through a portion of or entirely through the article 10. The opening 1224 may be formed within the absorptive matrix 12 during the molding process or may be formed in the article 10 using a mechanical tool to form the opening 1224. The opening 1224 may be configured for placement of a light source, such as an light emitting diode or other light source, within the article 10.

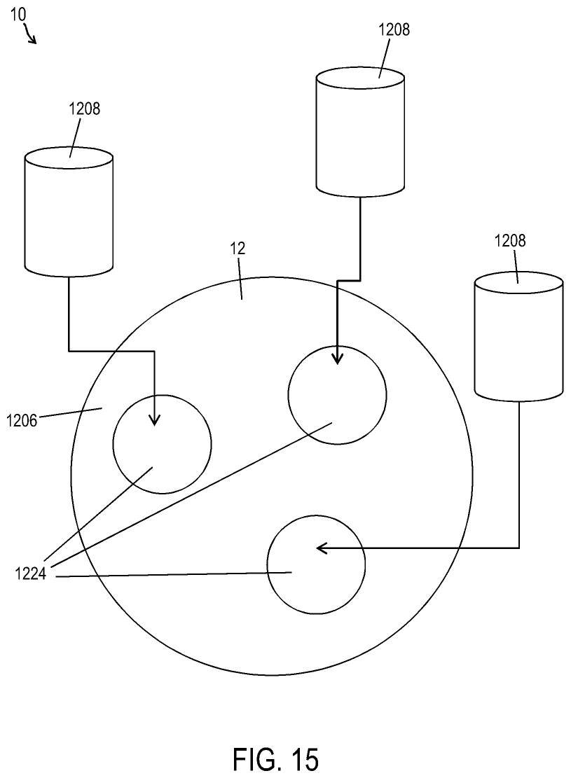

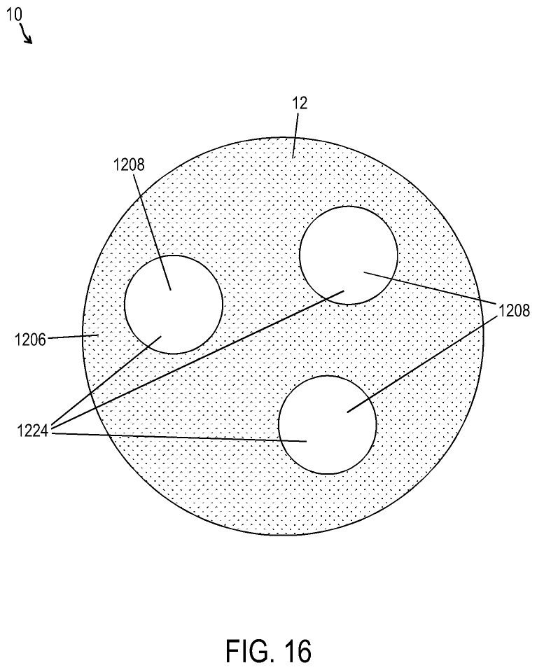

In further embodiments, multiple types of pulp materials 12 may be included in article 10. As one example, one or more openings 1224 may form a receptacle for the insertion of other absorptive matrices 12 or other materials or objects. In some embodiments, as best illustrated in FIGS. 15-16, the absorptive matrix 12 may be molded having a uniform first porosity without porosity zones 1202 but with at least one opening 1224. This opening 1224 may be shaped to receive another absorptive matrix 12 that is molded having a uniform second porosity without porosity zones 1202 and having a shape that substantially conforms to the shape and dimensions of the opening 1224. Once the second absorptive matrix 12 is inserted into the opening 1224, the article 10 may then comprise different porosity zones 1202 resulting from the different porosities of the other absorptive matrices 12. Additional openings 1224 may be included with the article 10, and more absorptive matrices 12 with additional different porosities may be inserted to form a plurality of porosity zones 1202. In further embodiments, other items such as scented rods of spiral wound paper, may be inserted into the openings 1224. Thus, the openings 1224 may serve as a way to replenish the volatile composition 24 within the article 10 by removing older base materials 12 or scented rods from which the scent has been depleted, and replacing them with new ones. In some examples, the absorptive matrix 12 (and/or the resultant article 10) may include inclusions based on other materials where the other materials are mixed into the absorptive matrix 12 before or during the forming/molding process (and/or are inserted into openings 1224). In some embodiments, the other materials are plant-based materials including, for example, flower petals. In some cases, the plant based materials are dried lavender, rose petals, or any other appropriate material. The other materials may also include minerals, rocks, shells, charcoal, and/or other inorganic decorative objects.

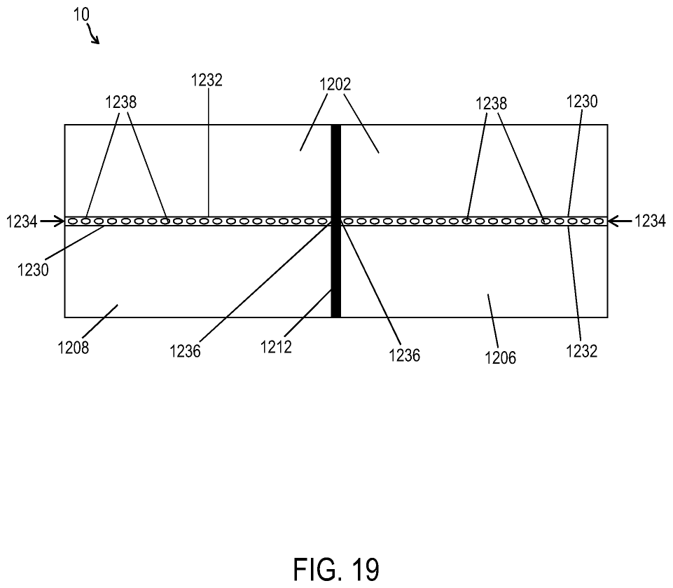

In other embodiments, as best illustrated in FIG. 19, a capillary structure 1230 may be incorporated into the dividers 1212 and/or may be a separate structure that is added to the mold 1204 prior to or during the absorptive matrix material addition. This capillary structure 1230 may comprise a length of tubing 1232 having one open end 1234 accessible from an outer surface of the absorptive matrix 12 and an opposing end 1236 terminating within the body of the absorptive matrix 12. The opposing end 1236 may be connected to the divider 1212 to suspend the capillary structure 1230 within the mold 1204 during the absorptive matrix material addition and molding process.

In certain embodiments, the capillary structure 1230 may comprise separate tubing extending through each zone 1206, 1208. The tubing may further comprise a series of small apertures 1238 along its length. The capillary structure 1230 may be used to reintroduce a volatile composition 24 into the zones 1206, 1208 once the concentration is depleted. The volatile composition 24 is introduced through the open end 1234 and disperses into the zones 1206, 1208 via the apertures 1238. Each zone 1206, 1208 may receive a different volatile composition 24 and/or the re-fill design allows for the volatile compositions 24 to be replaced with different scents as desired.

In certain embodiments, the article 10 may be combined with at least one energy source, including but not limited to a heating element (such as a warmer bowl or plate, electrical plug-in, chemical warmer pack, candle, light source, heating element system, and any other heat generating object, wherein the source of the energy is solar, battery, chemical, electrical, or any other suitable source of energy) and/or a wind element (such as a fan, blower, air circulation vent, bladeless fan, and any other air movement object, wherein the source of the energy is solar, battery, chemical, electrical, or any other suitable source of energy).

The article 10 may be combined with the energy source in a variety of manners. A variety of energy sources that are attached and/or placed in close proximity to articles containing volatile compositions are described in U.S. Publication No. 2015/0217016, the entire contents of which is incorporated herein by reference.

In some embodiments, the article 10 may be positioned within a warmer bowl or plate, wherein the article 10 is heated through contact with the surface of the warmer bowl. The surface of the warmer bowl or plate 1004 produces heat in a range of approximately 90° F. to 250° F. In further embodiments, a chemical warmer pack may be attached or positioned adjacent to the article 10.

In these embodiments, the backing layer 1222 may be configured to serve as a contact surface between the article 10 and the warmer bowl. To improve the efficiency of heat transfer between the article 10 and the warmer bowl, the backing layer 1222 may be formed of a conductive material such as tin, copper, aluminum, or other suitable metallic materials. In other embodiments (e.g., as described below in the context of scent producing assembly 2000), there is a gap between the article 10 and an energy source. In some of these embodiments, a backing layer 1222 would not be present or would be removed before operation of the device.

According to some embodiments, the article 10 may be shaped into a light shade or screen, which is positioned around and/or near an incandescent light bulb. For example, the article 10 may be positioned as a screen for a night light or a shade for small decorative lights. The article 10 may also be configured as a lamp shade or screen for larger bulbs.

The heat generated by the energy source may heat the volatile composition 24 within the article 10 so as to facilitate its release, and the wind generated by the energy source may create an air flow over the article 10, which facilitates dispersion of the volatile composition 24. Some examples related to the release/dispersion of volatile composition 24 are described in more detail below in the context of scent producing assembly 2000.

As described above, the density of the article 10 affects the amount of liquid fragrance that can be absorbed. In some embodiments, after the volatile composition 24 (or the combination of volatile composition 24 and the oil soluble dye) is added, the overall article 10 is approximately 30%-60% liquid (by weight). Some examples of the articles 10 may have 40% liquid by weight while other articles 10 may have 50% liquid by weight. In some embodiments, the article 10 has an internal reservoir capable of receiving up to 5-15 g of volatile composition 24 (or the combination of volatile composition 24 and the oil soluble dye). In some embodiments, the internal reservoir of the article 10 is capable of receiving up to 9 g of volatile composition 24 (i.e., the maximum liquid capacity). In some embodiments, the article 10 is designed to absorb approximately ⅔ of maximum liquid capacity. In some embodiments, the article 10 is designed to absorb approximately 6 g of volatile composition 24. The article 10 may also have at least one geometric feature (e.g., a channel, a recess, or any other relevant feature) that affects the amount of volatile composition 24 that is absorbed.

B. Volatile Composition

The volatile composition 24 may include, but is not limited to fragrances, flavor compounds, odor-eliminating compounds, aromatherapy compounds, natural oils, water-based scents, odor neutralizing compounds, and outdoor products (e.g., insect repellent).

As used herein, “volatile substance” refers to any compound, mixture, or suspension of compounds that are odorous, or any compound, mixture, or suspension of compounds that cancel or neutralize odorous compounds, such as any compound or combination of compounds that would produce a positive or negative olfactory sense response in a living being that is capable of responding to olfactory compounds, or that reduces or eliminates such olfactory responses.

A volatile composition as used herein comprises one or more volatile substances, and is generally a composition that has a smell or odor, which may be volatile, which may be transported to the olfactory system of a human or animal, and is generally provided in a sufficiently high concentration so that it will interact with one or more olfactory receptors.

A fragrance may comprise an aroma or odorous compound, mixture or suspension of compounds that is capable of producing an olfactory response in a living being capable of responding to olfactory compounds, and may be referred to herein as odorant, aroma, or fragrance. A fragrance composition may include one or more than one of the fragrance characteristics, including top notes, mid notes or heart, and dry down or base notes. The volatile composition 24 may comprise other diluents or additives, such as solvents or preservatives.

Examples of volatile compositions 24 useful in the present invention include, but are not limited to esters, terpenes, cyclic terpenes, phenolics, which are also referred to as aromatics, amines and alcohols. Further examples include, but are not limited to furaneol 1-hexanol, cis-3-Hexen-1-ol, menthol, acetaldehyde, hexanal, cis-3-hexenal, furfural, fructone, hexyl acetate, ethyl methylphenylglycidate, dihydrojasmone, wine lactone, oct-1-en-3-one, 2-Acetyl-1-pyrroline, 6-acetyl-2,3,4,5-tetrahydropyridine, gamma-decalactone, gamma-nonalactone, delta-octalactone, jasmine, massoia lactone, sotolon ethanethiol, grapefruit mercaptan, methanethiol, 2-methyl-2-propanethiol, methylphosphine, dimethylphosphine, methyl formate, nerolin tetrahydrothiophene, 2,4,6-trichloroanisole, substituted pyrazines, methyl acetate, methyl butyrate, methyl butanoate, ethyl acetate, ethyl butyrate, ethyl butanoate, isoamyl acetate, pentyl butyrate, pentyl butanoate, pentyl pentanoate, isoamyl acetate, octyl acetate, myrcene, geraniol, nerol, citral, lemonal, geranial, neral, citronellal, citronellol, linalool, nerolidol, limonene, camphor, terpineol, alpha-ionone, terpineol, thujone, benzaldehyde, eugenol, cinnamaldehyde, ethyl maltol, vanillin, anisole, anethole, estragole, thymoltrimethylamine, putrescine, diaminobutane, cadaverine, pyridine, indole and skatole. Most of these are organic compounds and are readily soluble in organic solvents, such as alcohols or oils. Fragrance includes pure fragrances, such as those including essential oils, and are known to those skilled in the art. Water-based odorous compounds and other odorous compositions are also contemplated by the present invention.

Fragrance oils as olfactory-active compounds or compositions usually comprise many different perfume raw materials. Each perfume raw material used differs from another by several important properties including individual character and volatility. By bearing in mind these different properties, and others, perfume raw materials may be blended to develop a fragrance oil with an overall specific character profile. To date, characters are designed to alter and develop with time as the different perfume raw materials evaporate from the substrate and are detected by the consumer. For example, perfume raw materials which have a high volatility and low substantivity are commonly used to give an initial burst of characters such as light, fresh, fruity, citrus, green, or delicate floral to the fragrance oil, which are detected soon after application. Such materials are commonly referred to in the field of fragrances as “top notes.” By way of a contrast, the less volatile, and more substantive, perfume raw materials are typically used to give characters such as musk, sweet, balsamic, spicy, woody or heavy floral to the fragrance oil, which may also be detected soon after application, but also last far longer. These materials are commonly referred to as “middle notes” or “base notes.” Highly skilled perfumers are usually employed to carefully blend perfume raw materials so that the resultant fragrance oils have the desired overall fragrance character profile. The desired overall character is dependent both upon the type of composition in which the fragrance oil will finally be used and also the consumer preference for a fragrance.

In addition to the volatility, another important characteristic of a perfume raw material is its olfactory detection level, otherwise known as the odor detection threshold (ODT). If a perfume raw material has a low odor detection threshold, only very low levels are required in the gas phase, or air, for it to be detected by the human, sometimes as low as a few parts per billion. Conversely, if a perfume raw material has a high ODT, larger amounts or higher concentrations in the air of that material are required before it can be smelled by the consumer. The impact of a material is its function of its gas phase or air concentration and its ODT. Thus, volatile materials, capable of delivering large gas-phase concentrations, which also have low ODTs, are considered to be impactful. To date, when developing a fragrance oil, it has been important to balance the fragrance with both low and high volatility raw materials, as the use of too many high volatility materials may lead to a short lived, overwhelming scent. As such, the levels of high odor impact perfume raw materials within a fragrance oil have traditionally been restricted.

As used herein, the term “fragrance oil” relates to a perfume raw material, or mixture of perfume raw materials, that are used to impart an overall pleasant odor profile to a composition, preferably a cosmetic composition. As used herein, the term “perfume raw material” relates to any chemical compound which is odorous when in an un-entrapped state. For example, in the case of pro-perfumes, the perfume component is considered to be a perfume raw material, and the pro-chemistry anchor is considered to be the entrapment material. In addition, “perfume raw materials” are defined by materials with a C log P value preferably greater than about 0.1, more preferably greater than about 0.5, even more preferably greater than about 1.0. As used herein the term “C log P” means the logarithm to base 10 of the octanol/water partition coefficient. This can be readily calculated from a program called “C LOG P,” which is available from Daylight Chemical Information Systems Inc., Irvine Calif., USA. Octanol/water partition coefficients are described in more detail in U.S. Pat. No. 5,578,563.

Examples of residual “middle and base note” perfume raw materials include, but are not limited to ethyl methyl phenyl glycidate, ethyl vanillin, heliotropin, indol, methyl anthranilate, vanillin, amyl salicylate, coumarin. Further examples of residual perfume raw materials include, but are not limited to, ambrox, bacdanol, benzyl salicylate, butyl anthranilate, cetalox, ebanol, cis-3-hexenyl salicylate, lilial, gamma undecalactone, gamma dodecalactone, gamma decalactone, calone, cymal, dihydro iso jasmonate, iso eugenol, lyral, methyl beta naphthyl ketone, beta naphthol methyl ether, para hydroxylphenyl butanone, 8-cyclohexadecen-1-one, oxocyclohexadecen-2-one/habanolide, florhydral, intreleven aldehyde.

Examples of volatile “top note” perfume raw materials include, but are not limited to anethol, methyl heptine carbonate, ethyl aceto acetate, para cymene, nerol, decyl aldehyde, para cresol, methyl phenyl carbinyl acetate, ionone alpha, ionone beta, undecylenic aldehyde, undecyl aldehyde, 2,6-nonadienal, nonyl aldehyde, octyl aldehyde. Further examples of volatile perfume raw materials include, but are not limited to phenyl acetaldehyde, anisic aldehyde, benzyl acetone, ethyl-2-methyl butyrate, damascenone, damascone alpha, damascone beta, flor acetate, frutene, fructone, herbavert, iso cyclo citral, methyl isobutenyl tetrahydro pyran, isopropyl quino line, 2,6-nonadien-1-ol, 2-methoxy-3-(2-methylpropyl)-pyrazine, methyl octine carbonate, tridecene-2-nitrile, allyl amyl glycolate, cyclogalbanate, cyclal C, melonal, gamma nonalactone, c is 1,3-oxathiane-2-methyl-4-propyl.

Other useful residual “middle and base note” perfume raw materials include, but are not limited to eugenol, amyl cinnamic aldehyde, hexyl cinnamic aldehyde, hexyl salicylate, methyl dihydro jasmonate, sandalore, veloutone, undecavertol, exaltolide/cyclopentadecanolide, zingerone, methyl cedrylone, sandela, dimethyl benzyl carbinyl butyrate, dimethyl benzyl carbinyl isobutyrate, triethyl citrate, cashmeran, phenoxy ethyl isobutyrate, iso eugenol acetate, helional, iso E super, ionone gamma methyl, pentalide, galaxolide, phenoxy ethyl propionate.

Other volatile “top note” perfume raw materials include, but are not limited to benzaldehyde, benzyl acetate, camphor, carvone, borneol, bornyl acetate, decyl alcohol, eucalyptol, linalool, hexyl acetate, iso-amyl acetate, thymol, carvacrol, limonene, menthol, iso-amyl alcohol, phenyl ethyl alcohol, alpha pinene, alpha terpineol, citronellol, alpha thuj one, benzyl alcohol, beta gamma hexenol, dimethyl benzyl carbinol, phenyl ethyl dimethyl carbinol, adoxal, allyl cyclohexane propionate, beta pinene, citral, citronellyl acetate, citronellal nitrile, dihydro myrcenol, geraniol, geranyl acetate, geranyl nitrile, hydroquinone dimethyl ether, hydroxycitronellal, linalyl acetate, phenyl acetaldehyde dimethyl acetal, phenyl propyl alcohol, prenyl acetate, triplal, tetrahydrolinalool, verdox, cis-3-hexenyl acetate.

In certain embodiments, the volatile composition 24 may comprise a fragrance component having a release rate ranging from 0.001 g/day to 2.0 g/day. The formulation of the fragrance may comprise any suitable combination of top, mid, and base note components.

In certain embodiments, the absorptive matrix 12 may be infused with more than one volatile composition 24 that is paired with a suitable zone 1206, 1208 within the absorptive matrix 12 to achieve a blended release rate designed to optimize the “top note” and “middle and base note” release rates.

As discussed above, the porosity (which may be controlled by fiber compactness, infusion of gas or gas-forming materials, refining, additives, or any other porosity-controlling method described above) may affect the uptake or load amount of the volatile composition 24, or may affect the rate of release of the volatile composition 24. For example, high porosity zone 1206, which has a lower fiber compactness, will provide an easier release of the volatile composition 24 because there are larger air passages between the fibers. Thus, a volatile composition 24 comprising mostly “middle and base note” components may be incorporated into the high porosity zone 1206 to provide an earlier release of the “middle and base note” components.

In contrast, low porosity zone 1208, which has a higher fiber compactness, will provide a more controlled release of the volatile composition 24 because the network of air passages through the fibers is tighter and more complex. Thus, a volatile composition 24 comprising mostly “top note” components may be incorporated into the low porosity zone 1208 to provide a slower release of the “top note” components.

In other words, the absorptive matrix 12 may be engineered with a plurality of zones, each zone having a uniquely designed pulp porosity that correlates to the desired release rate of the particular notes within the different volatile compositions 24.

In some embodiments, the design may be to create a simultaneous and sustained release of all notes, which may be optimized by pairing “top notes” with lower porosity zones, “middle notes” with medium porosity zones, and “base notes” with higher porosity zones.

In other embodiments, the design may be to create a staggered release of different scents over time, which may be optimized by reversing the pairing described above. In other words, the absorptive matrix 12 may include a pairing of “top notes” with higher porosity zones 1202, “middle notes” with medium porosity zones 1202, and “base notes” with lower porosity zones 1202.

The test results described in Example 2 demonstrate that an absorptive matrix 12 having a density of 0.36 g/mL generates a different release profile of a volatile composition with high and low molecular weight compounds, when compared to a absorptive matrix 12 having a density of 0.24 g/mL. In the fragrance industry, high molecular weight compounds are categorized as “base note” compounds, and low molecular weight compounds are categorized as “top note” volatile compounds.

Specifically, for samples containing only “base note” compound methyl cedryl ketone (“MCK”) volatile composition 24, the lower density absorptive matrix samples released approximately 12 times more “base note” MCK than the higher density absorptive matrix samples.

For samples containing both “top note” compound ethyl acetate volatile composition 24 and “base note” compound methyl cedryl ketone (“MCK”) volatile composition 24, the lower density absorptive matrix samples and the higher density absorptive matrix samples released the “base note” MCK at similar rates, while the lower density absorptive matrix samples released approximately 15 times more “top note” ethyl acetate than the higher density absorptive matrix samples.

Finally, the lower density absorptive matrix samples showed a faster release rate for all volatile compositions 24 over the higher density absorptive matrix samples.

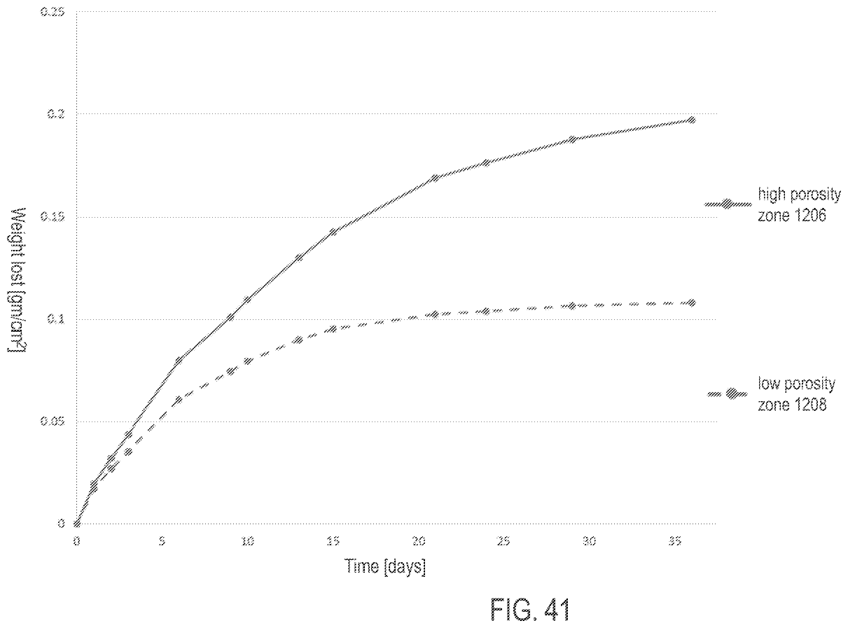

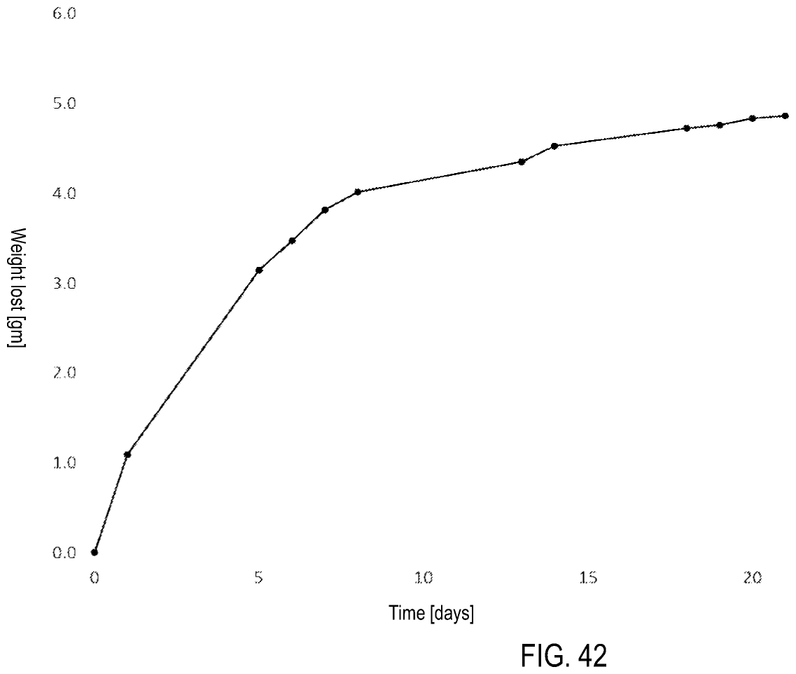

FIG. 41 shows weight loss data for high porosity zone 1206 and low porosity zone 1208, which is generated by measuring the cumulative amount of volatile composition released over time from each zone. Because, as described above, high porosity zone 1206 has a lower density (higher porosity) and thus can absorb more liquid compared to the low porosity zone 1208, the high porosity zone 1206 exhibits a greater weight loss (per surface area). FIG. 41 also illustrates that the rate of the weight loss for low porosity zone 1208 reduces faster than the rate of the weight loss for high porosity zone 1206. FIG. 42 shows an example of the cumulative weight loss for an article 10 over a 21 day period. While the period referenced is 21 days, other periods of longer or shorter duration may be employed when measuring the cumulative weight loss.

### EXAMPLES

### Example 1. Synthesis of Absorptive Matrix

Pulp material (15 g; southern hardwood; Sulfatate-H-J grade; Rayonier Performance Fibers, LLC) was added to a blender cup. A solution containing (i) colloidal silica (5 g; Snowtex®-O (silica 20% wt/wt in water); Nissan Chemical America Corporation), (ii) starch (5 g; Maltrin QD® M500 Maltodextrin NF; Grain Processing Corporation), (iii) baking powder (1 g; Clabber Girl Corporation), and (iv) water (221.5 g) was added to the blender cup. The content in the blender cup was blended to form a consistent pulp slurry, followed by removal of 100 g of excess solution. The final pulp slurry was added to a silicone mold, where the shape of the mold is a cylinder with dimensions 1.8 cm diameter, 1.3 cm height (volume: 3.31 cm³). The amount of pulp slurry used to create a varying density pulp cylinder is provided in Table 1.

TABLE 1
Pulp mass and density of pulp cylinder matrix

| | Pulp slurry | Pulp dry | Density (g |
|---|---|---|---|
| | mass (g) | mass (g) | pulp/cm³) |
| High density pulp | 10 | 1.2 | 0.36 |
| cylinder | | | |
| Low density pulp | 6 | 0.8 | 0.24 |
| cylinder | | | |

### Example 2. Headspace Gas Chromatography/Mass Spectrometry (GC/MS) Valuation of Release of High and Low MW Ingredients from an Absorptive Matrix

The amount of release of a top note or base note volatile ingredient from the absorptive matrix was evaluated using the standard method ASTM D4526-12 Standard for Determination of Volatiles in Polymers by Static Headspace Gas Chromatography. Headspace GC/MS experiments were carried out on Agilent instruments: headspace model 7697A, GC model 7850A, and MS model 5975C. The top note and base note ingredients selected are common ingredients used in all types of olfactive compositions in the fragrance industry. Ethyl acetate (CAS 141-78-6; MW 88.1 g/mol) is the top note ingredient that was tested, and methyl cedryl ketone (CAS 32388-5-9; MW 246.4 g/mol) is the base note ingredient that was tested. The base note ingredient represents the high end of the molecular weight spectrum for volatile ingredients, and the top note ingredient represents the low end of the molecular weight spectrum for volatile ingredients.

TABLE 2
Headspace GC/MS results demonstrating impact of packing density in absorptive
matrix 12 on release profile of olfactive volatile compositions.

| | Absorptive | | | | | Amount |
|---|---|---|---|---|---|---|
| | matrix | Compound | GC/MS peak | GC/MS peak | Amount EA | MCK |
| | density | injected | area | area | detected | detected |
| Sample | (g/mL) | (7 μL each) | (EA) | (MCK) | (%) | (%) |
| EA | NA | EA | 1191399736 | NA | 100 | NA |
| control | | | | | | |
| MCK | NA | MCK | NA | 1437276114 | NA | 100 |
| control | | | | | | |
| 1 | 0.36 | EA | Below limit | NA | not detected | NA |
| 2 | 0.36 | MCK | NA | 21830631 | NA | 1.52 |
| 3 | 0.36 | EA/MCK | Below limit | 3915890 | not detected | 0.27 |
| 4 | 0.24 | EA | Below limit | NA | not detected | |
| 5 | 0.24 | MCK | NA | 270003206 | NA | 18.79 |
| 6 | 0.24 | EA / MCK | 186196145 | 4025104 | 15.63 | 0.28 |

EA = ethyl acetate;
MCK = methyl cedryl ketone;
NA = not applicable

### Example 3. Illustration of Fiber Density in Absorptive Matrix 12 by Epoxy Embedding and Thin Section Imaging

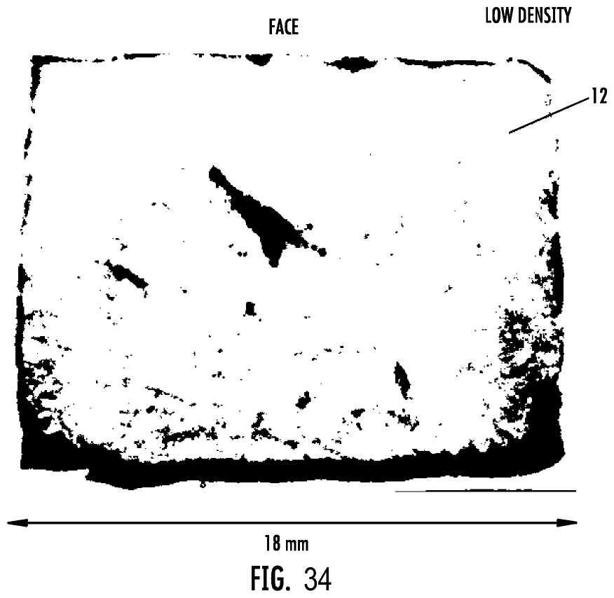

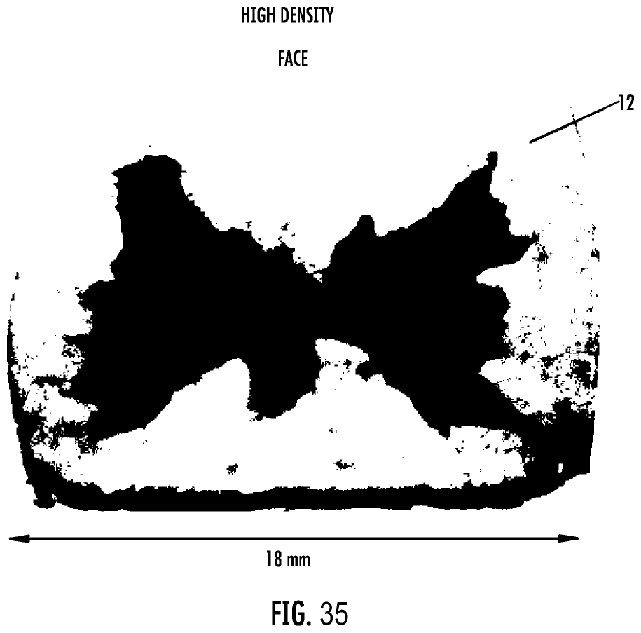

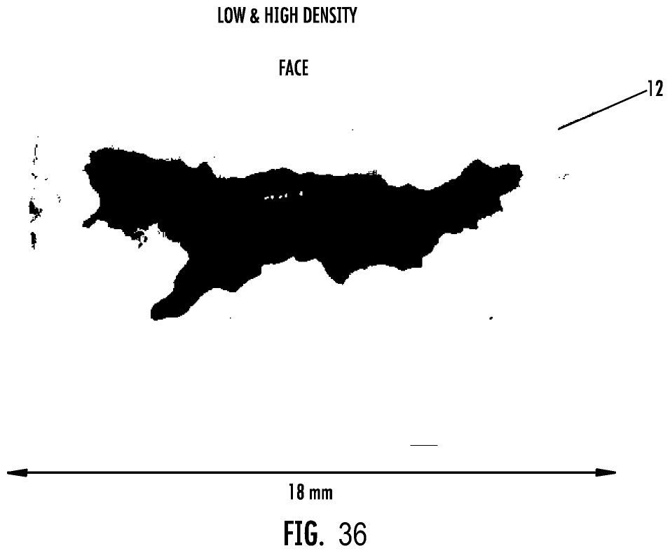

Samples of a three-dimensional pulp object with a high density (0.36 g/mL) and a three-dimensional pulp object with a low density (0.24 g/mL) absorptive matrix 12 were analyzed using Epoxy Embedding and Thin Section Imaging. Each sample was vacuum filled with Epofix cold mount epoxy resin distributed by Electron Microscopy Sciences. A thin section of each sample was cut with a saw blade and immersed in Cargill refractive index liquid (R.I.=1.572, which matches the R.I. of Epofix). Transmitted light imaging was then used to capture micrographs of the cross-sections of each sample, as may be seen in FIGS. 34, 35 and 36. The dark, spiked features at the centers of the samples indicate incomplete impregnation of the epoxy resin, which also indirectly indicates fiber density. For example, as may be seen in FIG. 35, the epoxy resin impregnation is less complete in the high density sample than in the low density sample shown in FIG. 39. Moreover, FIG. 36, which includes a sample of a three-dimensional pulp object with both high density and low density absorptive matrix 12, also illustrates less complete epoxy resin impregnation in the area with a higher density than in the area with a lower density. Additionally, in FIG. 35, the faint, gradual change in density from top to bottom in the high density sample, excluding the dark center, is an artifact caused by a change in thin section thickness, as the sample is wedge-shaped. However, the sample in FIG. 36, which includes both high density and low density absorptive matrices 12, has a uniform thickness, and thus the faintly darker upper half is indicative of the higher density absorptive matrix 12 in that area.

C. Modulating Coating or Additive

As used herein, “coating” or “additive” refers to any composition that may be applied using any suitable method to at least one of an outer surface of the article 10, to some or all surfaces of the absorptive matrix 12, and/or may be uniformly or non-uniformly distributed throughout the internal structure 20 of the base material 12 and/or the article 10. In cases of surface application, the coating may be applied so that the composition may or may not penetrate to at least some degree within the article 10 and/or the base material 12.

Modulating coating or additive 14 may be applied to at least one outer surface 16 of the base material 12 and/or to the article 10, and may be applied before or after loading of the volatile composition 24. In certain embodiments, the Modulating coating or additive 14 may penetrate into the internal structure 20 of the base material 12 to a certain level, which may vary depending on the porosity, processing methods, or other characteristics of the base material 12.

The modulating coating or additive 14 is designed to slow the release rate of the volatile composition 24 loaded into the internal structure 20 at higher concentration levels and accelerate the release rate of the volatile composition 24 at lower concentration levels in order to achieve a relatively steady release of volatile composition 24 over time. In some embodiments, the material of the modulating coating or additive 14 is mixed with the base material 12 before and/or during the molding/forming process for the article 10.

To explain the way that the modulating coating or additive 14 works to have this “hold/push” effect over a range of load levels of the volatile composition 24, it is necessary to explain the way in which the release rate of the volatile composition 24 is generated. The volatile composition 24 is loaded or absorbed into the internal structure 20 via the pores 22 until a sufficiently high load level is achieved within the internal structure 20 through various embodiments of loading methods, which are explained in detail below. The volatile composition 24 may be loaded or absorbed into the internal structure 20 before or after the modulating coating or additive 14 is applied.

The initially high load level of the volatile composition 24 within the internal structure 20 creates an internal force that causes the volatile composition 24 to diffuse or evaporate out of the internal structure 20 as quickly as possible to a region of lower concentration. As the load level of the volatile composition 24 decreases over time, the force that causes the diffusion or evaporation diminishes until there is no longer a force remaining (i.e., an equilibrium point is reached where the volatile composition 24 no longer diffuses or evaporates out of the internal structure 20). The equilibrium point is usually higher than 0% concentration, which causes some of the volatile composition 24 to become trapped within the pores 22 of the internal structure 20.

In conventional applications, such as in U.S. Publication No. 2011/0262377, a coating may be applied to form a layer that slows or retards the rapid release of a volatile composition at higher concentration levels. These conventional coatings typically include substances that trap some of the volatile composition within the coating layer, which slows down the rate of release through the coating. However, because the coating only serves as a barrier or “speed bump” to slow down the rate of release of the volatile composition, the release will eventually stop once the concentration of volatile composition within the internal structure reaches equilibrium (i.e., a level where there is no longer a sufficient concentration to drive the volatile composition through the coating layer, thus allowing some of volatile composition to remain trapped within the coating layer and/or within the internal structure).

The modulating coating or additive 14 comprises both a barrier substance 26 and a hygroscopic substance 28. In particular, in most embodiments, the modulating coating or additive 14 comprises substances that do not chemically interact with the volatile composition 24 itself.

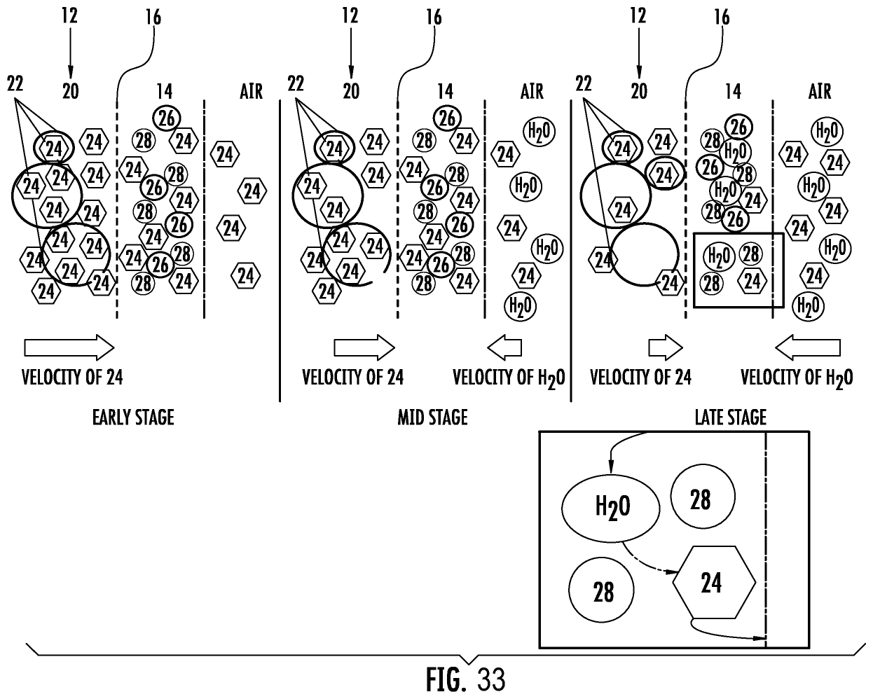

In these embodiments, when the modulating coating or additive 14 is applied to the outer surface 16 of the internal structure 20, at the higher concentration levels of the volatile composition 24 within the internal structure 20, the barrier substance 26 forms a barrier or “speed bump” to slow down the rate of release of the volatile composition 24 through the modulating coating or additive 14. At these higher initial concentration levels, as illustrated in the early stage section of FIG. 33, the hygroscopic substance 28 does not play a role in modulating the release rate of the volatile composition 24 (i.e., does not absorb any water into the modulating coating or additive 14) because the concentration of the volatile composition 24 within the internal structure 20 is sufficiently high to force a certain amount of the volatile composition 24 to release through the modulating coating or additive 14 at a rate that effectively blocks any water from being attracted into the modulating coating or additive 14 by the hygroscopic substance 28.

As the concentration level of the volatile composition 24 within the internal structure 20 slowly diminishes, as illustrated in the mid stage section of FIG. 33, the concentration of the volatile composition 24 within the internal structure 20 is still sufficiently high to continue to force some of the volatile composition 24 out of the modulating coating or additive 14 at a reduced rate of release.

One hypothesis to explain the phenomenon observed in the late stage is that because there is a lower volume of the volatile composition 24 exiting the modulating coating or additive 14, the hygroscopic substance 28 begins to attract more water (typically in the form of water vapor) into the modulating coating or additive 14, whereupon the water adsorbs or absorbs to the hygroscopic substance 28 and begins to displace the volatile composition 24 that is trapped by the barrier substance 26 within the modulating coating or additive 14. This hypothesis is illustrated in the late stage section of FIG. 33, and is based on known physical properties of the hygroscopic substance 28 and the data showing higher release rates at the end of the product life cycle, as compared to the same product without the modulating coating or additive 14. Once displaced, the volatile composition 24 is released from the modulating coating or additive 14, thereby creating an aggregate rate of release of the volatile composition 24 that may approximate the rate of release driven by the higher load level of the volatile composition 24 alone.

As the load level of volatile composition 24 continues to drop to a level that can no longer drive the volatile composition 24 out of the modulating coating or additive 14, the hygroscopic substance 28 continues to pull more and more water into the modulating coating or additive 14. That water continues to displace the trapped volatile composition 24, effectively forcing the displaced volatile composition 24 to be released from the modulating coating or additive 14. For a period of time in the late stage, the rate of release of the volatile composition 24 due to water displacement driven by the hygroscopic substance 28 may approximate the rate of release driven by the higher load level of the volatile composition 24 alone and/or may approximate the aggregate rate of release driven by both the higher load level of the volatile composition 24 and water displacement driven by the hygroscopic substance 28. As a result, where conventional coatings that contain only barrier substances 26 may have stopped releasing volatile compositions once the equilibrium point of the concentration is reached within the internal structure 20, the modulating coating or additive 14 continues to provide a relatively constant release of the volatile composition 24.

An alternate hypothesis to explain the phenomenon observed in the late stage is that the water that is brought into the modulating coating or additive 14 by the hygroscopic substance 28 may act to degrade the barrier substance 26, which would also allow for release of the volatile composition 24 trapped within the modulating coating or additive 14 and within the internal structure 20 of the base material 12.

In any event, the test results demonstrate that the modulating coating or additive 14 generates an improved release profile of the volatile composition 24 over the aromatic life cycle of the article 10, depending on the porosity of the internal structure 20 of the base material 12 and the volatility levels of the volatile composition 24. Eventually, the concentration of the volatile composition 24 within the internal structure 20 and the amount trapped by the barrier substances 26 within the modulating coating or additive 14 will reach such a low point that the amount of volatile composition 24 released on a daily basis by the modulating coating or additive 14 will eventually decline to zero. A series of examples supporting and explaining this process are provided in U.S. Publication No. 2016/0089468, the entire contents of which are incorporated herein by reference.

In certain embodiments, the barrier substance 26 may comprise maltodextrin (e.g. Maltrin). In other embodiments, the barrier substance 26 may include, but is not limited to other dextrins, other film-forming polysaccharides, other carbohydrates (mono-, di-, tri-, etc.), natural unmodified starch, modified starch, any starch appropriate for use in papermaking, as well as combinations of starch types, dextrin types, and combinations of starches and dextrins. In certain embodiments, the barrier substance 26 may include, but not is limited to additives such as insolubilizers, lubricants, dispersants, defoamers, crosslinkers, binders, surfactants, leveling agents, wetting agents, surface additives, rheology modifiers, non-stick agents, and other coating additives.

In certain embodiments, the hygroscopic substance 28 may comprise silica (e.g. silica nanoparticles). In other embodiments, the hygroscopic substance 28 may include, but is not limited to other hygroscopic reagents, activated charcoal, calcium sulfate, calcium chloride, molecular sieves, or other suitable water absorbing materials.

The weight ratio of the barrier substance 26 to the hygroscopic substance 28 may range from 99:1 to 1:99, and all ranges therein between. In certain embodiments, weight ratio of the barrier substance 26 to the hygroscopic substance 28 may further range from 25:75 to 75:25. In yet other embodiments, the weight ratio of the barrier substance 26 to the hygroscopic substance 28 may be approximately 50:50.

In certain embodiments, the particle size of the hygroscopic substance 28 is determined in part by the amount of surface area needed to attract enough water to counteract the drop in release rate due to a reduction in the load level of the volatile composition 24. The hygroscopic substance 28 is also configured so that it will attract water vapor, rather than liquid water. As a result, the diameter of the particle size of the hygroscopic substance 28 may range from 0.001 μm-1 μm, and all ranges therein between, and may further range from 1 nm-100 nm, which will attract the appropriate amount of water vapor molecules, as well as provide a more even coating.

In certain embodiments, the hygroscopic substance 28 may have a surface charge range that ensures interaction with the barrier substances 26. For example, in the case of silica, the surface charge ranges from −10 mV to −4000 mV, as measured by Zeta potential, which is a highly anionic point charge. When the silica is mixed with the maltodextrin before coating, the maltodextrin may group around the silica particles, which may further assist with the barrier formation within the modulating coating or additive 14.

In certain embodiments, the modulating coating or additive 14 may provide a more consistent release rate of the volatile composition 24. The consistency (variance) may be measured by the following formula.

Variance_{(Weight-loss ratio)}=First day weight-loss value/Last day weight-loss value

A benefit of the modulating coating or additive 14 is to reduce the variance within a ratio range of 1 to 20 over a life cycle of the article, which in certain embodiments may be at least 30 days, 45 days, or 60 days, but could be longer or shorter as needed or desired.

In certain embodiments, the modulating coating or additive 14 may be used in combination with the porosity zones 1202 described above. For example, the modulating coating or additive 14 may be applied to the external surfaces of the absorptive matrix 12 or may only be applied to the external surfaces of the low porosity zone 1208 to further enhance the regulating effect of the low porosity/high density design of that zone for top note volatile components 24.

An additional benefit of the modulating coating or additive 14 is the structural reinforcement that the modulating coating or additive 14 provides to the absorptive matrix 12, particularly for the high porosity zones 1206. In some embodiments, the modulating coating or additive 14 may only be applied to the external surfaces of the high porosity zone 1206 to provide additional stability to those high porosity zones 1206, even if the coating may also temper the release rate of base note volatile compositions 24 from the high porosity zones 1206.

D. Additional Treatment of the Base Material and/or Article

The base material 12 may be converted into the article 10, which may occur before or after the modulating coating or additive 14 and/or the volatile composition 24 are applied.

In further embodiments, the article 10 may comprise a three-dimensional structure with varying shapes and sizes including but not limited to a cylindrical disk, cylinder, tree, wreath, globe, orb, pine cone, star, bell, stocking, bag, gift box, snowman, penguin, reindeer, santa claus, heart, angel, basket, flower, butterfly, leaf, face, bird, fish, mammal, reptile, pyramid, cone, snowflake, other polygonal shape, fan blade or a portion thereof. The article 10 may have one or more flat surfaces, concave surfaces, convex surfaces, surfaces that are smooth, and/or surfaces that contain complex geometry (e.g., peaks and valleys), or any other suitable surface configuration.

In certain embodiments, the article 10 may comprise a spiral wound paper. The spiral winding process allows for the paper to be the same or different for each layer formed by winding the paper one complete revolution around the axis of the structural component. For example, the article 10 may comprise a rod shape, formed by winding the absorptive matrix 12 around a vertical axis, so that a rod having a length longer than its diameter is formed. Each layer formed by the complete revolution of the paper matrix around the axis may be referred to as a ply. For example, a 10 ply rod may have from one to ten different characteristics for each ply of the rod. Characteristics may include but are not limited to absorbance, tensile strength density, pH, porosity, and polarity of the base material 12, and the type of paper or internal structure 20.

The modulating coating or additive 14 may be applied to the absorptive matrix 12 before or after application of the volatile composition 24.

The modulating coating or additive 14 may be applied to absorptive matrix 12 after it has been removed from the mold 1204 and/or after it has been formed into the article 10.

For example, the modulating coating or additive 14 may be applied to the absorptive matrix 12 and/or the article 10 via a dip method where the three-dimensional article 10 is placed within a volume of modulating coating or additive 14 for a specified amount of time, then removed and allowed to dry. The dip method may also be used with two-dimensional versions of the article 10. The add-on level may range from 0.1% to 10% by weight.

In other embodiments, the modulating coating or additive 14 may be applied to the absorptive matrix 12 and/or the article 10 via an infusion method with the add-on infusion ranging from 1% to 20% by weight, and, in certain embodiments, may further range from 10% to 20% by weight.

In yet other embodiments, the modulating coating or additive 14 may be applied to absorptive matrix 12 and/or the article 10 via spray treatment.

The volatile composition 24 may be applied to the base material 12 before or after application of the modulating coating or additive 14, as described above. For example, the volatile composition 24 may be applied by placing the base material 12 and/or the article 10 in intimate contact with the volatile composition 24 for a period of time. The pre-absorbed volatile composition 24 may be in any physical state, such as liquid, solid, gel, or gas. For convenience, a liquid volatile composition 24 prior to absorption is described, but this is not intended to be limiting. The interaction time may depend on the concentration or type of volatile composition 24 being applied to the base material 12 and/or the article 10, and/or how strong or intense of a volatile composition 24 release is desired, and/or the type of base material 12. The saturation time (interaction time) may range from less than one minute to a several hours, to several days. The base material 12 and/or the article 10 may be pre-treated prior to exposure to the volatile composition 24. For example, the base material 12 and/or the article 10 may be placed in a drying oven to remove any residual moisture. Further method steps comprise pressure treating and/or vacuum treating the base material 12 and/or the article 10. After treatment, the base material 12 and/or the article 10 may be dried, for example by rubbing or patting dry, and/or by other methods known for drying a surface, and/or may be left to air dry. Drying steps may be used before or after other steps described herein.

In some embodiments, a method for applying the volatile composition 24 to the base material 12 and/or to the article 10 comprises combining the volatile composition 24 and the base material 12 and/or the article 10 in a container and applying a pressure above atmospheric pressure on the volatile composition 24 and base material 12 and/or the article 10. Pressure may be applied in a range from about 1 psi to about 40 psi, from about 5 psi to about 30 psi, or from about 10 psi to about 20 psi, at about 5 psi, at about 10 psi, at about 15 psi, at about 20 psi, at about 25 psi, at about 30 psi, at about 35 psi, at about 40 psi, and/or at pressures therein between. The pressure may be applied for a period of time from about 1 minute to about 10 hours, for about 30 minutes, for about 1 hour, for about 2 hours, for about 3 hours, for about 4 hours, for about 5 hours for about 6 hours, for about 7 hours, for about 8 hours, for about 9 hours, for about 10 hours, or longer if needed to apply sufficient amounts of the volatile composition 24 to the base material 12 and/or the article 10 to achieve a desired load of the volatile composition 24 to the base material 12 and/or the article 10 or release of the volatile composition 24 from the base material 12 and/or the article 10. Appropriate pressures and times for a particular embodiment can be determined by one skilled in the art based on the identities and characteristics of the particular volatile composition 24 and base material 12 and/or article 10.

In certain embodiments, a method for applying the volatile composition 24 comprises combining the volatile composition 24 and base material 12 and/or the article 10 in a container and applying a vacuum below atmospheric pressure to the volatile composition 24 and the base material 12 and/or the article 10. Vacuum may be applied in a range from 0.001 mm Hg to about 700 mm Hg, or from about 5 Kpa to about 35 kPa, from about 10 Kpa to about 25 kPa, from about 20 Kpa to about 30 kPa, from about 15 Kpa to about 25 kPa, from about 25 Kpa to about 30 kPa, at about 5 kPa, at about 6 kPa, at about 7 kPa, at about 8 kPa, at about 9 kPa, at about 10 kPa, at about 15 kPa, at about 16 kPa, at about 17 kPa, at about 18 kPa, at about 19 kPa, at about 20 kPa, at about 22 kPa, at about 24 kPa, at about 26 kPa, at about 28 kPa, at about 30 kPa, and vacuums therein between. The vacuum may be applied for a period of time from about 1 minute to about 10 hours, for about 30 minutes, for about 1 hour, for about 2 hours, for about 3 hours, for about 4 hours, for about 5 hours for about 6 hours, for about 7 hours, for about 8 hours, for about 9 hours, for about 10 hours, or longer if needed to apply sufficient amounts of the volatile composition 24 to the base material 12 and/or the article 10 to achieve a desired load of the volatile composition 24 to the base material 12 and/or the article 10 or release of the volatile composition 24 from the base material 12 and/or the article 10.

In yet other embodiments, the method may comprise pressure and vacuum steps. The volatile composition 24 and the base material 12 and/or the article 10 may be combined and undergo vacuum treatment and pressure treatment, in no particular order. For example, the volatile composition 24 and the base material 12 and/or the article 10 may be combined in a container in an air-tight apparatus and a vacuum of 20 mm Hg to 80 mm Hg may be applied for about 1 minute to 10 hours. Pressure treatment of 1 psi to 40 psi may be applied for about 1 minute to about 10 hours and the time and amount of vacuum or pressure treatment may vary and depend upon the amount of volatile composition 24 to be loaded in the base material 12 and/or the article 10, the type of base material 12 used, the intended use of the article 10, and other characteristics of the article 10.

In certain embodiments, the base material 12 and/or the article 10 may be pre-treated with colorants, followed by treatment with the modulating coating or additive 14. Colorants may include natural and synthetic dyes, water-resistant dyes, oil-resistant dyes, oil soluble dyes, and combinations of water- and oil-resistant dyes. Colorants may be selected based on the composition of the base material 12, and is well within the skill of those in the art. Suitable water-resistant colorants include oil soluble colorants and wax soluble colorants. Examples of oil soluble colorants include Pylakrome Dark Green and Pylakrome Red (Pylam Products Company, Tempe Ariz.). Suitable oil-resistant colorants include water soluble colorants. Examples of water soluble colorants include FD&C Blue No. 1 and Carmine (Sensient, St. Louis, Mo.). A Lake type dye may also be used. Examples of Lake dyes are Cartasol Blue KRL-NA LIQ and Cartasol Yellow KGL LIQ (Clariant Corporation, Charlotte, N.C.). Pigments may also be used in coloring the base material 12 and may be added during or after the manufacture of the base material 12. Such coloring or dying methods are known to those skilled in the art, and any suitable dyes, pigments, or colorants are contemplated by the present invention. Colorants may be used to affect the overall surface charge of the silica or other hygroscopic substance 28 to enhance the interaction with the coating.

E. Solvent-Free Fragrance Dispenser

According to certain embodiments, the article 10 is formed of all-natural, biodegradable, recyclable, compostable and sustainably sourced materials, such as wood pulp. These materials are combined with all-natural biodegradable, recyclable, compostable performance boosters, such as silica, starch, and baking soda. The product is then treated with fragrance, such as 100% pure fragrance in the form of all-natural essential oils and/or other responsibly selected and harvested fragrance materials.

Specifically, in some cases, the article 10 does not include a chemical solvent. Chemical solvents minimize the amount of fragrance that can be used (by as much as 85%) and compromise duration. Furthermore, chemical solvents may have a chemical overtone that is difficult to entirely overcome with perfume.

F. Scent Producing Assemblies

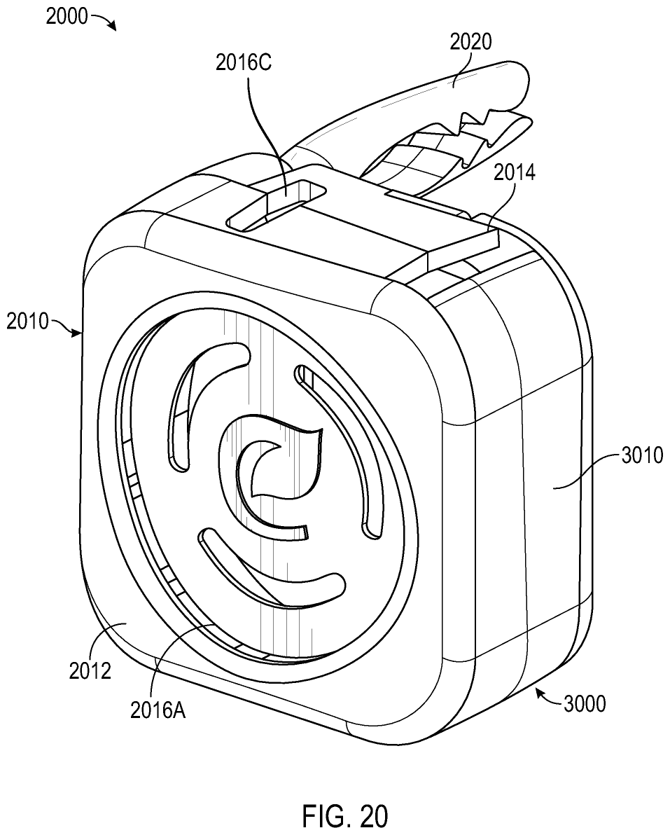

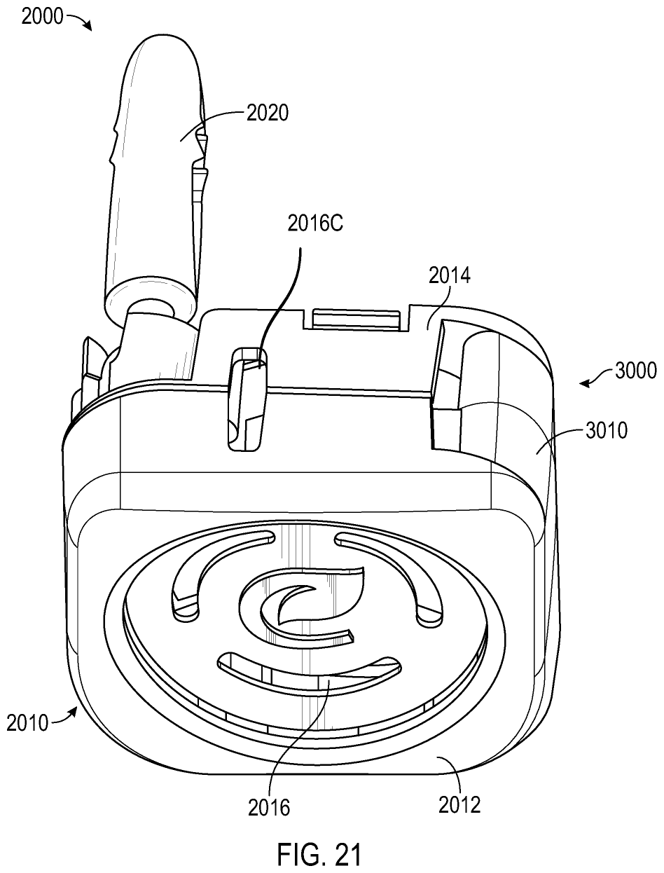

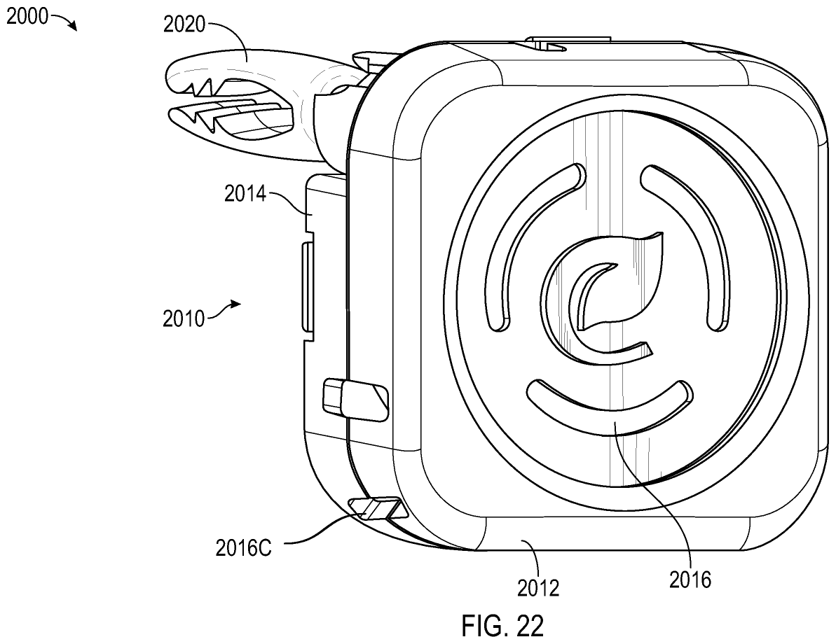

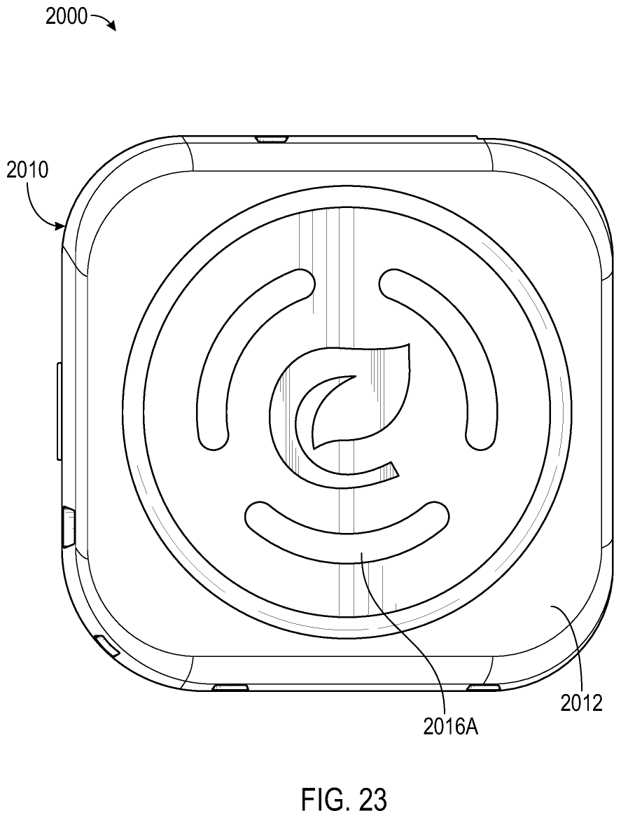

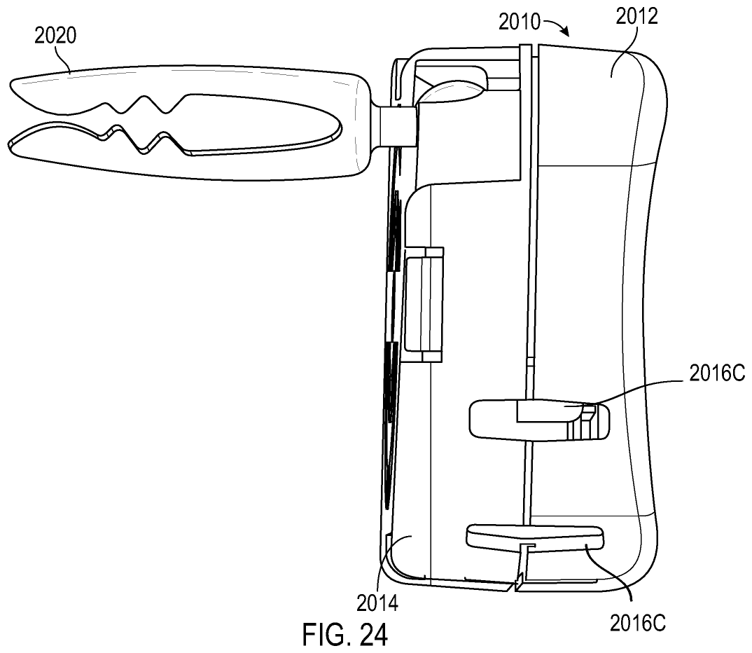

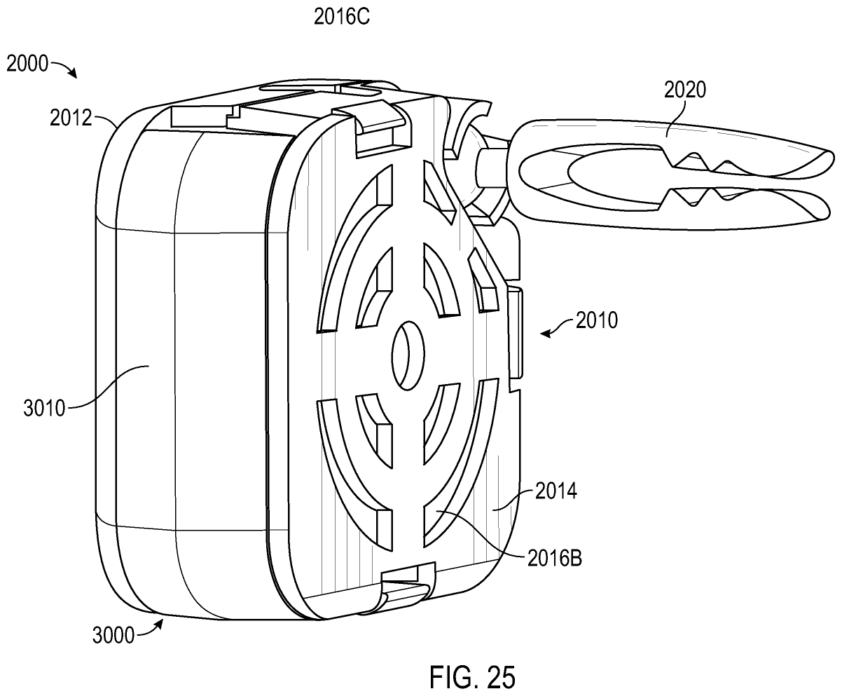

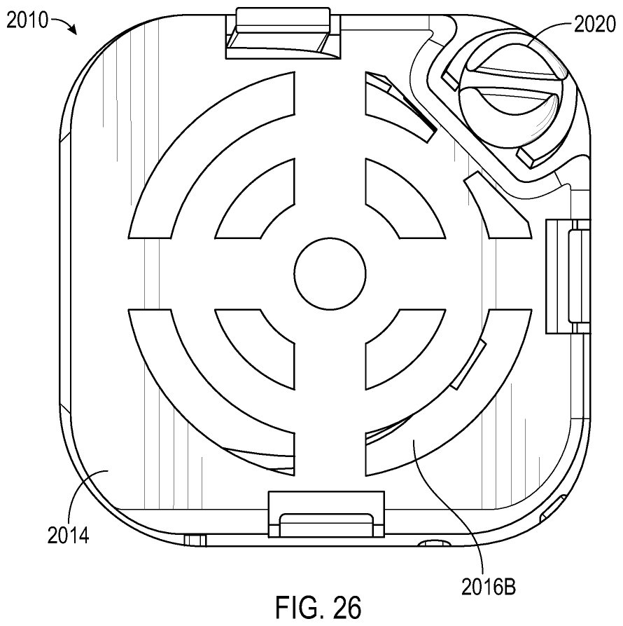

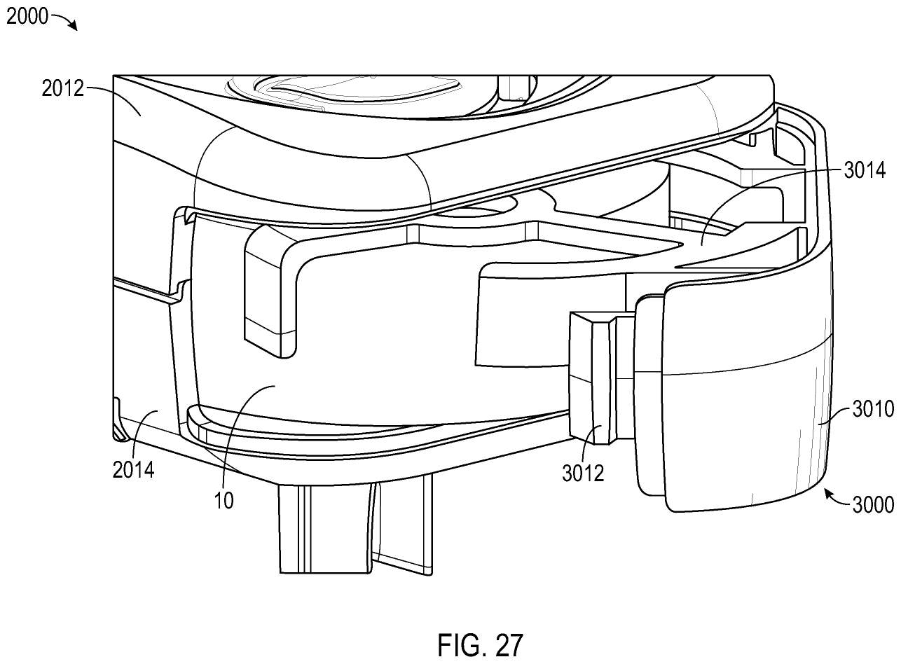

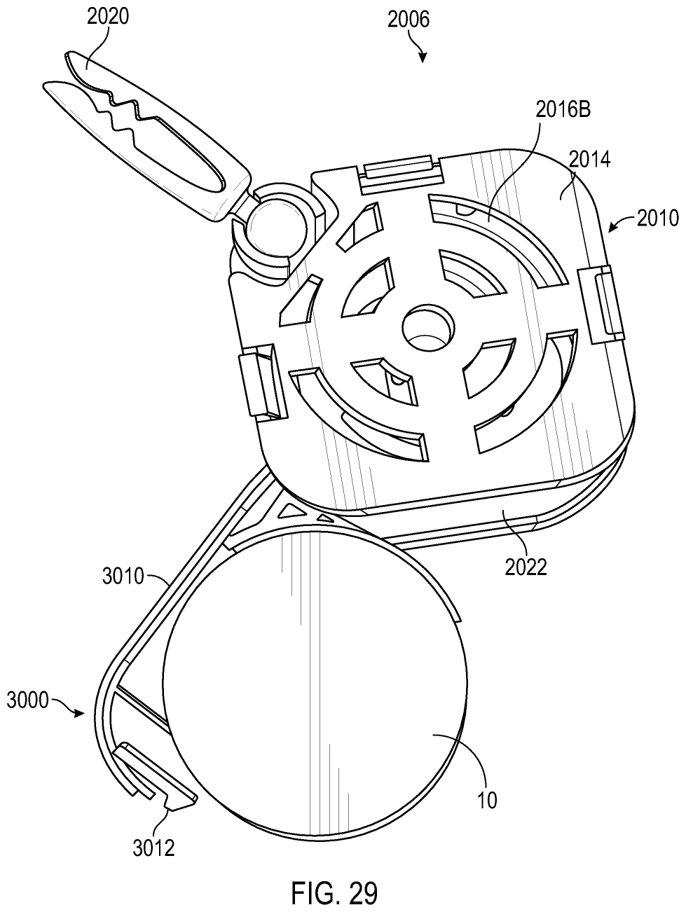

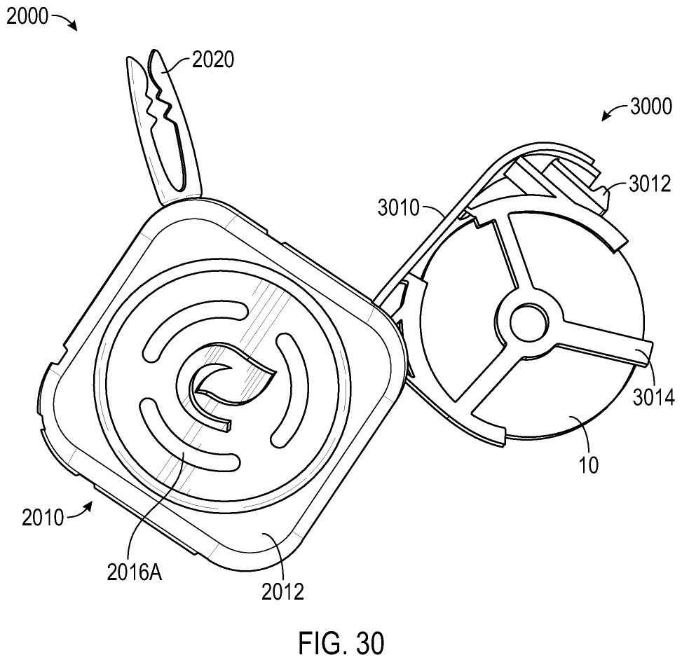

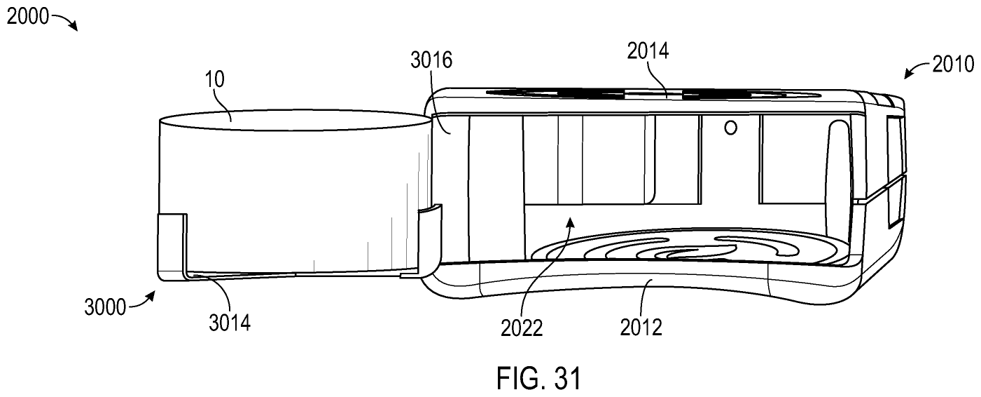

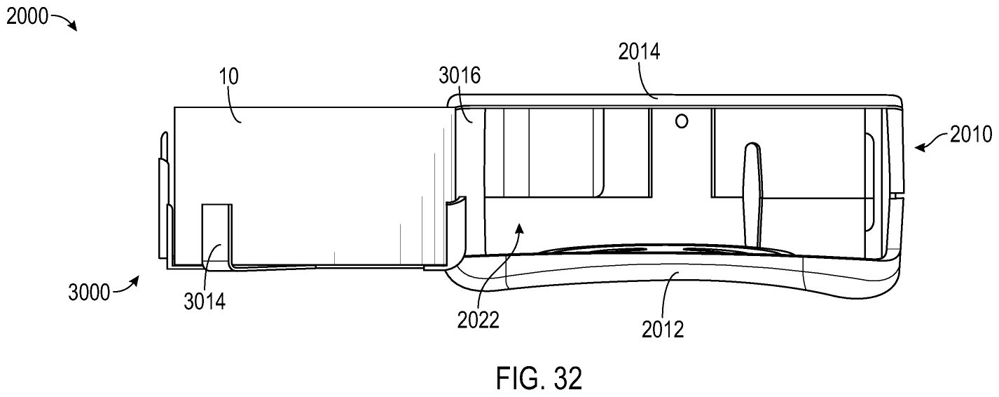

According to certain embodiments, as illustrated in FIGS. 20-32, a scent producing assembly 2000 may include the article 10, which includes the volatile composition 24, and a housing 2010. In some embodiments, the scent producing assembly 2000 may be a vent clip device; however, in other embodiments, the scent producing assembly 2000 may be a standalone device, such as for a tabletop or floor-standing, and/or any other appropriate configuration.

The housing 2010 of the scent producing assembly 2000 may include a housing exterior and a housing interior. The housing interior may be hollow. The housing exterior may include a front cover 2012 and a rear cover 2014. The front cover 2012 may include at least one front cover opening 2016A. The at least one front cover opening 2016A may be a single slit or aperture; alternatively, the front cover 2012 may include multiple front cover openings 2016A formed by multiple slits or apertures. Similarly, the rear cover 2014 may include at least one rear cover opening 2016B similar to the at least one front cover opening 2016A. As described with the respect to the at least one front cover opening 2016A, the at least rear cover opening 2016B may be a single slit or aperture or, alternatively, multiple rear cover openings 2016B formed by multiple slits or apertures. Moreover, the housing 2010 may further include at least one side opening 2016C disposed on a side of the housing 2010. For example, the at least one side opening 2016C may be disposed on a side of the housing 2010 the front cover 2012 and the rear cover 2014. The at least one side opening 2016C may be on a top side of the housing 2010, a bottom side of the housing 2010, a lateral side of the housing 2010, or any combination thereof. Moreover, as described above with respect to the at least one front opening 2016A and the at least one rear opening 2016B, the at least one side opening 2016C may be formed of a single slit or aperture, or alternatively, may be formed of multiple slits or apertures. In some embodiments, the at least one front cover opening 2016A the at least one rear cover opening 2016B and the at least one side opening 2016C permit air to flow through the housing 2010, thereby allowing dispersion of the volatile composition 24 out of the housing 2010 and into a surrounding environment.

In some embodiments, the housing 2010 is shaped in a square configuration. However, the housing 2010 may be configured in any suitable shape based on a user's needs and/or desires, including, but not limited to, a circle, a triangle, a rectangle, an ellipse, an octagon, a hexagon, or any other suitable shape.

The housing 2010 may have any suitable dimensions determined by a user's needs and/or wants. For example, the dimensions may include a length and width between 0.75 to 3 inches, and a depth between 0.3 to 1.5 inches. As described above, as the housing 2010 may be of any suitable shape based on a user's needs and/or desires, the length and the width of the housing 2010 may be the same or different depending on the particular shape of the housing 2010.

As described above, the article 10 may be formed of materials such that the article 10 is recyclable and/or biodegradable. Rather than being formed of a non-recyclable and/or non-biodegradable material, wherein the article 10 must be discarded in an environmentally unfriendly manner, the article 10 may be formed as a self-contained scented article 10 that can be easily replaced and/or discarded based on a user's needs and/or desires. When a user replaces or discards the article 10, the article 10 may be discarded in an environmentally friendly manner (e.g., recycle, compost). Thus, for example, the article 10 may be replaced and recycled rather than being thrown away as a single use product. Similarly, the scent producing assembly 2000 may be recyclable and/or biodegradable. The article 10 may be inserted within the scent producing assembly 2000 thereby forming a self-contained scented article, in which a user may easily refill or replace the article 10 within the scent producing assembly 2000 without the use of non-recyclable materials. Thus, the scent producing assembly 2000 may be reused and refilled as opposed to being thrown away as a single use product.

In some embodiments, the scent producing assembly 2000 includes an attachment device 2020 coupled the housing exterior for securing or holding the scent producing assembly 2000. The attachment device 2020 may be coupled to the rear cover 2014, as illustrated in FIGS. 20-22, 24-26, and 29-30. However, in some embodiments, the attachment device 2020 may be attached to the front cover 2012. Moreover, while only one attachment device 2020 is illustrated, the scent producing assembly 2000 may include multiple attachment devices 2020 attached to the rear cover 2014, the front cover 2012, or both.

The attachment device 2020 may be any suitable attachment member, including, but not limited to, an alligator clip, hole, a protrusion, a hook, a male/female clip, an anchor, a hook and loop fastener, a magnet, a pin, a screw-type fastener, and/or any other appropriate object for securing the scent producing assembly 2000. Moreover, the attachment device 2020 may be coupled to the housing 2010 by any suitable manner, including, but not limited to ball and socket joint, hook and loop fastener, gluing, welding, magnets, or any other suitable attachment means. In some embodiments, the attachment device 2020 may also be integrally formed with the housing 2010.

In some embodiments, as illustrated in FIGS. 20-21, 25, and 27-32, the scent producing assembly 2000 includes a receptacle 3000 having an outer edge 3010 and a holding portion 3014. The receptacle 3000 may include a first end coupled to the housing 2010 at a single pivot point 3016 and a second end removably coupled to the housing 2010. The second end may include a locking mechanism 3012 (to be described in further detail). The holding portion 3014 may be shaped to receive and hold the article 10 in place. The holding portion 3014 may formed of a shape that complements the shape of the article 10. For example, the holding portion 3014 may include a bottom surface having a plurality of upward extending portions forming a recess between the plurality of upward extending portions. The article 10 may sit within the recess of the holding portion 3014 such that the article 10 remains stationary during movement of the receptacle 3000.

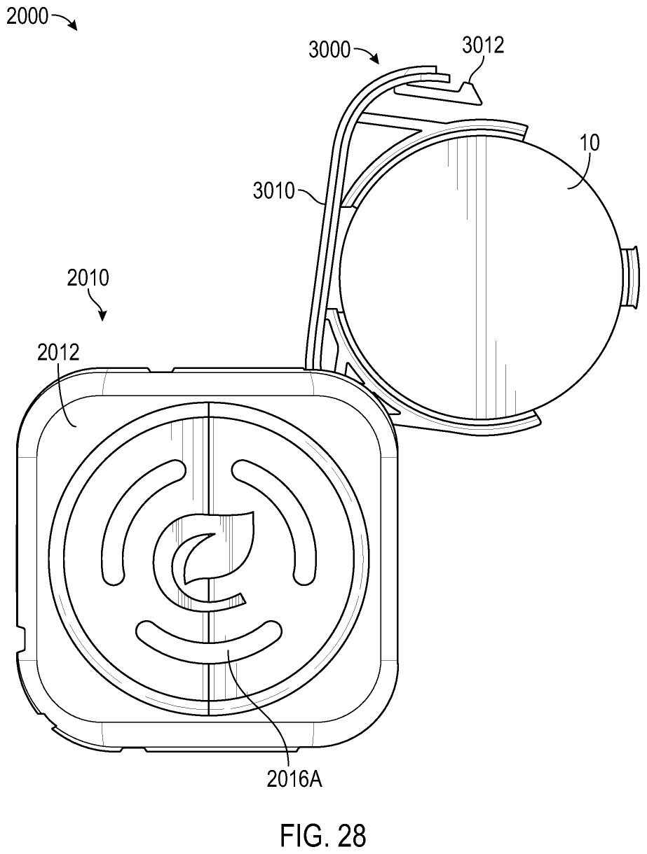

In some embodiments, the receptacle 3000 has a stowed position and a deployed position. As illustrated in FIGS. 20-21 and 25, when the receptacle 3000 is in the stowed position, the holding portion 3014 may be contained within the interior of the housing 2010 and the outer edge 3010 may form an exterior edge of the housing 2010. The outer edge 3010 may be of a shape and size that complements the housing 2010, such that, when the receptacle 3000 is in the stowed position, the outer edge 3010 is flush with the housing 2010. In the deployed position, as illustrated in FIGS. 28-32, the receptacle 3000 may be positioned outside of the housing 2010. Moreover, in the deployed position, the outer edge 3010 may no longer form a side of the housing 2010, thereby exposing an opening 2022 of the housing 2010 and allowing access to an interior of the housing 2010. In the deployed position, a user may remove the article 10 from and/or replace the article 10 within the receptacle 3000.

The first end of the receptacle 3000 may be coupled to the housing 2010 via the pivot point 3016. The receptacle 3000 may be integrally formed with the housing 2010, or, alternatively, may be attached via any suitable means, including, but not limited to, screwing, welding, gluing, hook and loop, snap fit, magnets, or any other suitable attachment means. When moving between the stowed and deployed positions, the receptacle 3000 may rotate about the pivot point 3016, such that the receptacle 3000 moves along an arcuate path. In particular, the first end of the receptacle 3000 may rotate about the pivot point 3016, while the second end of the receptacle 3000 moves along the arcuate path to the deployed position. During movement, the holding portion 3014 of the receptacle 3000 may move through the opening 2022 of the housing 2010 in order to move the article 10 from within the interior of the housing to the exterior of the housing. The locking mechanism 3012 of the receptacle 3000 may be configured to lock the receptacle 3000 in the stowed position such that the receptacle 3000 is not inadvertently dislodged and moved to the deployed position. The locking mechanism 3012 may be integrally formed with the receptacle 3000. For example, the locking mechanism 3012 may include, but is not limited to, a notch, an extension, a protrusion, or any other suitable member. Alternatively, the locking mechanism 3012 may be an additional structure attached to the receptacle 3000. The locking mechanism 3012 may operate by any suitable means for locking the receptacle 3000 in the stowed position, for example, an interlocking connection, a snap fit connection, hook and loop, magnetic connection, or any other suitable locking means. Moreover, the locking mechanism 3012 may operate such that the locking mechanism 3012 locks the receptacle 3000 from within the interior of the housing 2010 (i.e., the locking mechanism 3012 interacts with the interior of the housing 2010 to lock the receptacle in the stowed position). Alternatively, the locking mechanism 3012 may operate such that the locking mechanism 3012 locks the receptacle 3000 on the housing exterior (i.e., the locking mechanism 3012 interacts with the exterior of the housing to lock the receptacle in the stowed position).

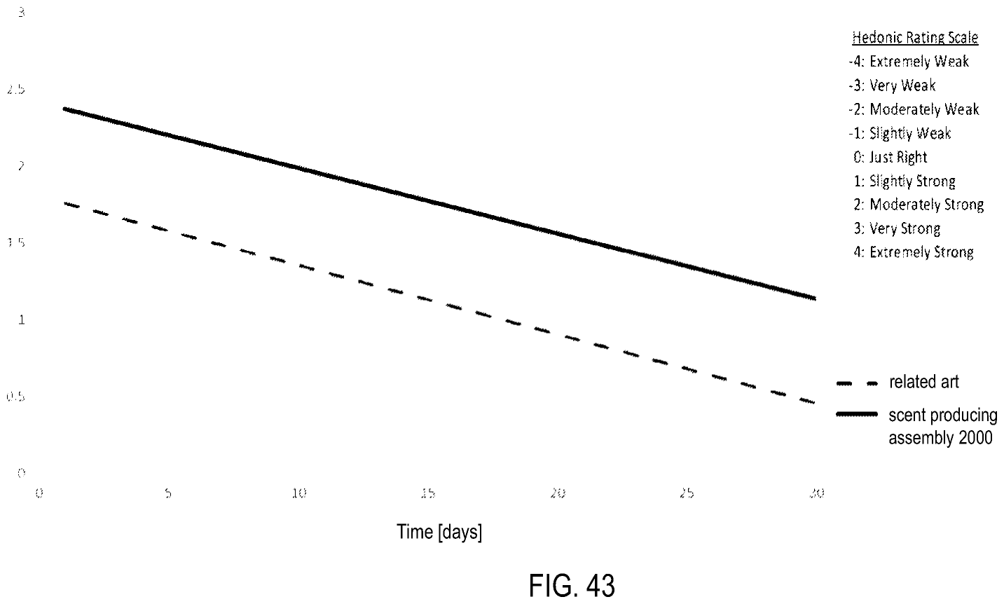

FIG. 43 shows hedonic test data for the perceived strength of the scent produced by the scent producing assembly 2000. The data for the scent producing assembly 2000 is compared to related art where the related art includes the same solid materials without an air gap 2062 between the energy source and the scent source. As shown in FIG. 43, the strength of the scent for the scent producing assembly 2000 and the related art both decline approximately linearly over a 30 day period. While the measurements in these examples were conducted over 30 days, 45 days, 60 days, or longer or shorter periods may be employed as needed or desired. However, the strength of the scent produced by the scent producing assembly 2000 is consistently greater than the strength of the scent produced by the related art.

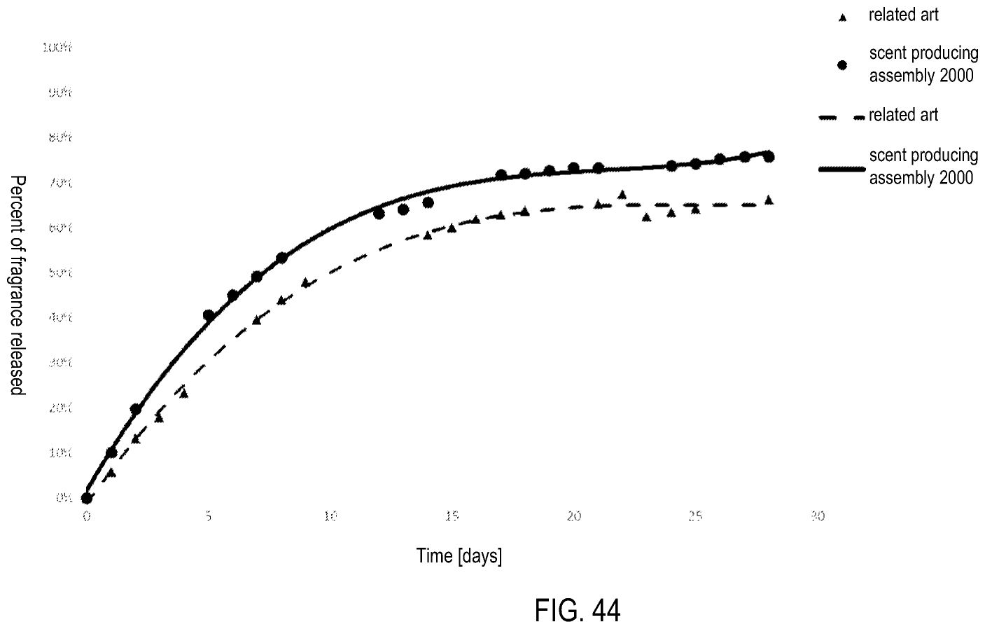

The scent producing assembly 2000 may also exhibit improvements in the amount of weight lost over a specified time period compared to the related art. FIG. 44 compares the weight lost (shown as the percentage of fragrance released) over a time period for the scent producing assembly 2000 compared to the related art. After 25-27 days, the related art (e.g., products that do not include an air gap 2062 between the energy source and the scent source) has released approximately 65% of the fragrance. In contrast, the scent producing assembly 2000, which does include an air gap 2062 between the energy source and the article, has released more than 75% of the fragrance after 25-27 days. There is a discernable difference between the amount of fragrance released from the scent producing assembly 2000 compared to the related art as soon as 1-2 days, which continuously increases.

The components of the scent producing assembly 2000 may be formed of materials including, but not limited to, polypropylene, polycarbonate, polyethylene terephthalate, acrylic, fluorinated polyethylene, polymers, graphite composite, polyester, nylon, thermoplastic, other plastic materials, wood, metal, or other suitable materials. These materials may be fire or flame resistant (or retardant). Moreover, the components of the scent producing assembly 2000 may be attached to one another via suitable fasteners, which include, but are not limited to, screws, magnets, bolts, rivets, or other mechanical or chemical fasteners.

In the following, further examples are described to facilitate understanding of aspects of the invention:

Example A. A scent producing assembly comprising:

a self-contained module comprising an absorptive matrix infused with a volatile composition;

a housing comprising a receptacle shaped to receive self-contained the module;

wherein the receptacle has a stowed position such that the receptacle is contained within an interior of the housing;

wherein the receptacle has a deployed position such that the receptacle is displaced outside of the housing; and

wherein, when the receptacle is moved between the stowed and deployed positions, the receptacle moves along an arcuate path.

Example B. The scent producing assembly of any of the preceding or subsequent examples, wherein the housing further comprises an opening along an edge; and

wherein during movement of the receptacle between the stowed and deployed positions, the receptacle moves through the opening.

Example C. The scent producing assembly of any of the preceding or subsequent examples, wherein, in the stowed position, an exterior surface of the receptacle forms an exterior edge of the housing at the opening.

Example D. The scent producing assembly of any of the preceding or subsequent examples, wherein the receptacle is attached to the housing via a single pivot point; and wherein, during movement between the stowed and deployed positions, the receptacle rotates about the pivot.

Example E. The scent producing assembly of any of the preceding or subsequent examples, wherein the receptacle further comprises a locking mechanism such that the receptacle is lockable in the stowed position.

Example F. The scent producing assembly of any of the preceding or subsequent examples, the self-contained module is formed of at least one of recyclable materials or biodegradable materials.

Example G. A scent producing assembly comprising:

a self-contained module comprising an absorptive matrix infused with a volatile composition;

a housing comprising:

a housing interior;

a housing exterior;

an opening formed along a side of the housing exterior;

a receptacle shaped to receive the self-contained module and having a stowed position and a deployed position;

wherein, in the stowed position, the receptacle is disposed within the housing interior;

wherein, in the deployed position, the receptacle is disposed outside of the housing interior; and

wherein, when the receptacle is moved between the stowed position and the deployed position, the receptacle moves through the opening along an arcuate path.

Example H. The scent producing assembly of any of the preceding or subsequent examples, wherein the receptacle comprises a first end and a second end; and wherein the first end is attached to the housing at a pivot point and the second end comprises an extension for locking the receptacle in the stowed position; and wherein during movement between the stowed and deployed positions, the receptacle pivots about the pivot point and the second end travels along the arcuate path.

Example I. The scent producing assembly of any of the preceding or subsequent examples, wherein the self-contained module is formed of at least one of recyclable materials or biodegradable materials.

Example J. The scent producing assembly of any of the preceding or subsequent examples, further comprising an attachment mechanism disposed on the housing exterior.

Example K. The scent producing assembly of any of the preceding or subsequent examples, wherein the housing exterior comprises openings on a first side and a second side such that air can freely pass through the housing.

Example L. The scent producing assembly of any of the preceding or subsequent examples, wherein the self-contained module is contained within the receptacle during movement between the stowed and deployed positions.

Example M. The scent producing assembly of any of the preceding or subsequent examples, wherein, when the receptacle is in the stowed position, an exterior surface of the receptacle forms an edge of the housing exterior.

A scent producing assembly comprising:

a self-contained module comprising an absorptive matrix infused with a volatile composition;

a housing comprising:
an interior wherein the interior is hollow;
a receptacle shaped to receive the self-contained module;

wherein the receptacle is connected to an edge of the housing at a pivot point;

wherein the self-contained module is moved between the interior of the housing and an exterior of the housing via the receptacle; and

wherein, during movement of the self-contained module between the interior of the housing and the exterior of the housing, the receptacle pivots about the pivot point and moves along an arcuate path.

Example O. The scent producing assembly of any of the preceding or subsequent examples, wherein the housing further comprises a first side and a second side; and

wherein each of the first side and the second side comprises at least one opening such that air freely passes through the housing.

Example P. The scent producing assembly of any of the preceding or subsequent examples, wherein the housing further comprises an opening along an edge between the first side and the second side; and

wherein the opening provides access to the interior of the housing.

Example Q. The scent producing assembly of any of the preceding or subsequent examples, wherein, when the receptacle is disposed within the interior of the housing, an outer edge of the receptacle forms an exterior edge of the housing.

Example R. The scent producing assembly of any of the preceding or subsequent examples, wherein, during movement of the self-contained module between the interior and exterior of the housing, the receptacle moves through opening.

Example S. The scent producing assembly of any of the preceding or subsequent examples, wherein the receptacle further comprises a locking mechanism at one end opposite the pivot point such that the receptacle is lockable when the self-contained module is disposed within the interior of the housing.

Example T. The scent producing assembly of any of the preceding or subsequent examples, further comprising an attachment means disposed on an exterior of the housing.

Different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications may be made without departing from the scope of the claims below.

1. A scent producing assembly comprising:
a self-contained module comprising an absorptive matrix infused with a volatile composition;
a housing comprising a receptacle shaped to receive self-contained the module; wherein the receptacle has a stowed position such that the receptacle is contained within an interior of the housing;
wherein the receptacle has a deployed position such that the receptacle is displaced outside of the housing; and
wherein, when the receptacle is moved between the stowed and deployed positions, the receptacle moves along an arcuate path.

2. The scent producing assembly of claim 1, wherein the housing further comprises an opening along an edge; and
wherein during movement of the receptacle between the stowed deployed positions, the receptacle moves through the opening.

3. The scent producing assembly of claim 2, wherein, in the stowed position, an exterior surface of the receptacle forms an exterior edge of the housing at the opening.

4. The scent producing assembly claim 1, wherein the receptacle is attached to the housing via a single pivot point; and
wherein, during movement between the stowed and deployed positions, the receptacle rotates about the pivot point.

5. The scent producing assembly of claim 1, wherein the receptacle further comprises a locking mechanism such that the receptacle is lockable in the stowed position.

6. The scent producing assembly of claim 1, wherein the self-contained module is formed of at least one of recyclable materials or biodegradable materials.

7. A scent producing assembly comprising:
a self-contained module comprising an absorptive matrix infused with a volatile composition;
a housing comprising:
a housing interior;
a housing exterior;
an opening formed along a side of the housing exterior;
a receptacle shaped to receive self-contained module and having a stowed position and a deployed position;
wherein, in the stowed position, the receptacle is disposed within the housing interior; wherein, in the deployed position, the receptacle is disposed outside of the housing interior; and
wherein, when the receptacle is moved between the stowed position and the deployed position, the receptacle moves through the opening along an arcuate path.

8. The scent producing assembly of claim 7, wherein the receptacle comprises a first end and a second end; and
wherein the first end is attached to the housing at a pivot point and the second end comprises an extension for locking the receptacle in the stowed position; and
wherein, during movement between the stowed and deployed positions, the receptacle pivots about the pivot point and the second end travels along the arcuate path.

9. The scent producing assembly of claim 8, wherein the self-contained module is formed of at least one of recyclable materials or biodegradable materials.

10. The scent producing assembly of claim 7, further comprising an attachment mechanism disposed on the housing exterior.

11. The scent producing assembly of claim 7, wherein the housing exterior comprises openings on a first side and a second side such that air can freely pass through the housing.

12. The scent producing assembly of claim 7, wherein the self-contained module is contained within the receptacle during movement between the stowed and deployed positions.

13. The scent producing assembly of claim 7, wherein, when the receptacle is in the stowed position, an exterior surface of the receptacle forms an edge of the housing exterior.

14. A scent producing assembly comprising:
a self-contained module comprising an absorptive matrix infused with a volatile composition;
a housing comprising:
an interior wherein the interior is hollow;
a receptacle shaped to receive the self-contained module;
wherein the receptacle is connected to an edge of the housing at a pivot point; wherein the self-contained module is moved between the interior of the housing and an exterior of the housing via the receptacle; and
wherein, during movement of the self-contained module between the interior of the housing and the exterior of the housing, the receptacle pivots about the pivot point and moves along an arcuate path.

15. The scent producing assembly of claim 14, wherein the housing further comprises a first side and a second side; and
wherein each of the first side and the second side comprises at least one opening such that air freely passes through the housing.

16. The scent producing assembly of claim 15, wherein the housing further comprises an opening along an edge between the first side and the second side; and
wherein the opening provides access to the interior of the housing.

17. The scent producing assembly of claim 16, wherein, when the receptacle is disposed within the interior of the housing, an outer edge of the receptacle forms an exterior edge of the housing.

18. The scent producing assembly of claim 16, wherein, during movement of the self-contained module between the interior and exterior of the housing, the receptacle moves through opening.

19. The scent producing assembly of claim 14, wherein the receptacle further comprises a locking mechanism at one end opposite the pivot point such that the receptacle is lockable when the self-contained module is disposed within the interior of the housing.

20. The scent producing assembly of claim 14, further comprising an attachment means disposed on an exterior of the housing.
